(19) United States
(12) Patent Application Publication
(10) Pub. No.: US 2026/0345729 A1
(43) Pub. Date: Nov. 5, 2026

(54) TISSUE DIFFERENTIATION FACTOR RELATED POLYPEPTIDES (TDFRPS) FOR THE TREATMENT OF MYOCARDIAL INJURY

(71) Applicant: Therapeutics By Design, LLC, Weston, MA (US)

(72) Inventor: William Dean Carlson, Weston, MA (US)

(73) Assignee: Therapeutics By Design, LLC, Weston, MA (US)

(21) Appl. No.: 19/119,646

(22) PCT Filed: Oct. 10, 2023
(86) PCT No.: PCT/US2023/034834

§ 371 Date: Apr. 9, 2025

Related U.S. Application Data
(60) Provisional application No. 63/414,747, filed on Oct. 10, 2022.

(51) Int. Cl. A61K 38/18; A61K 45/06; A61P 9/10

(52) U.S. Cl. CPC A61K 38/1841; A61K 45/06; A61P 9/10

(57) ABSTRACT

The present disclosure relates generally to methods of treating or preventing myocardial injury, or a condition associated with myocardial injury, in a mammal, wherein the method comprises administering at least one tissue differentiation factor related polypeptide (TDFRP) to the mammal, and wherein the administering is in an amount effective to treat or prevent myocardial injury in said mammal.

### RELATED APPLICATIONS

This application claims priority to U.S. Provisional Application No. 63/414,747, filed on Oct. 10, 2022, the contents of which is hereby incorporated by reference in its entirety.

### BACKGROUND

Any disorder that affects the heart muscle is called a cardiomyopathy. Cardiomyopathy can develop in people of all ages, races, and ethnicities, and often medical conditions or lifestyle habits can raise the risk of cardiomyopathy. Some types of cardiomyopathy are more common in certain age groups; arrhythmogenic cardiomyopathy is more common in teens and young adults; dilated cardiomyopathy is more common in adults between 20 and 60 years old; hypertrophic cardiomyopathy is more common in people in their 30s; takotsubo cardiomyopathy is more common in women after menopause. Some of the conditions that lead to cardiomyopathy include valvular heart disease, myocardial ischemia, pulmonary diseases, obstructive sleep apnea, inflammation (due to e.g., viral, bacterial, or fungal infection, or autoimmune disease), chemotherapy or radiation treatment for cancer, long term alcohol and drug abuse, heavy metal poisoning, nutritional issues, arrhythmias, constrictive pericarditis, diabetes, hemochromatosis, and complications of late-stage pregnancy. Other medical conditions that may lead to cardiomyopathy include Duchenne muscular dystrophy, sarcoidosis or amyloidosis, heart inflammation from endocarditis, myocarditis, or pericarditis, infections, such as viral hepatitis and HIV, obesity, diabetes, or other problems with the metabolic system.

Worldwide, about 15.9 million myocardial infarctions (MI) occurred in 2015 (Vos et al., (2015) Lancet. 388 (10053): 1545-1602). More than 3 million people had an ST elevation MI, and more than 4 million had an NSTEMI (non-ST elevation myocardial infarction) (White H D, Chew D P (2008) Lancet. 372 (9638): 570-84). STEMIs (ST elevation myocardial infarction occur about twice as often in men as women. About one million people have an MI each year in the United States. In the developed world, the risk of death in those who have had an STEMI is about 10% (Steg et al. (October 2012) European Heart Journal. 33 (20): 2569-619). Rates of MI for a given age have decreased globally between 1990 and 2010. In 2011, an MI was one of the top five most expensive conditions during inpatient hospitalizations in the US, with a cost of about $11.5 billion for 612,000 hospital stays (Torio C (August 2013). HCUP).

Myocardial infarction is a common presentation of coronary artery disease. The World Health Organization estimated in 2004, that 12.2% of worldwide deaths were from ischemic heart disease; with it being the leading cause of death in high- or middle-income countries and second only to lower respiratory infections in lower-income countries. Worldwide, more than 3 million people have STEMIs and 4 million have NSTEMIs a year. STEMIs occur about twice as often in men as women.

Rates of death from ischemic heart disease (IHD) have slowed or declined in most high-income countries, although cardiovascular disease still accounted for one in three of all deaths in the US in 2008. For example, rates of death from cardiovascular disease have decreased almost a third between 2001 and 2011 in the United States.

In contrast, IHD is becoming a more common cause of death in the developing world. For example, in India, IHD had become the leading cause of death by 2004, accounting for 1.46 million deaths (14% of total deaths) and deaths due to IHD were expected to double during 1985-2015. Globally, disability adjusted life years (DALYs) lost to ischemic heart disease are predicted to account for 5.5% of total DALYs in 2030, making it the second-most-important cause of disability (after unipolar depressive disorder), as well as the leading cause of death by this date.

Cardiomyopathy is often characterized by a decrease in the pumping function of the heart. The most common way to assess cardiac function is by measuring the ejection fraction (EF), which is the proportion of the blood volume that is effectively force into aortic circulation. A reduction in EF can be due to weakening of the ventral muscle from some type of injury. Cardiomyopathy can be caused by a number of conditions including, including but not limited to, valvular abnormalities, hypertension, collagen vascular disease, myocardial ischemia, myocardial infarction, toxins, anti-cancer therapies, congenital abnormalities, arrhythmias, diabetes and other types of injury. The underlying pathophysiology of cardiomyopathy is characterized by cardiomyocyte apoptosis, inflammation, and fibrosis (scarring). Loss of cardiac function results in increased stress on the heart and thus a worsening of the cardiomyopathic condition resulting in a spiral into congestive heart failure and death.

Treatments for cardiomyopathy generally address symptoms and causes: beta-blockers to relax stress by relaxing the heart, diuretics to remove excess fluid, spironolactone to reduce fluid and relax the heart, and pacemakers and implanted defibrillators to control arrhythmias. However, ultimately, cardiomyopathy is a condition of the heart muscle myocytes. If cardiomyocyte injury is allowed to progress, the result is apoptosis, necrosis (loss of heart muscle), inflammation and fibrosis (scarring and stiffening of the heart muscle), all of which result in loss of heart function resulting in increased mortality and morbidity.

Since cardiomyopathy encompasses a wide range of conditions, all resulting in loss of heart function, there exists a substantial need in the art for improved methods and compositions for treating a wide range of cardiac diseases and injuries characterized by cardiomyopathy.

### SUMMARY

Cardiac injury can be the result of myriad insults, but is mediated through a common pathophysiology including a apoptosis, inflammation, fibrosis, that leads to a syndrome called congestive heart failure or cardiomyopathy.

This present disclosure is based on the finding that tissue differentiation factor related polypeptides (TDFRPs) can be used to block and reverse the intracellular pathways in cardiomyocytes that lead to inflammation and apoptosis, thereby preventing necrosis and fibrosis. The present disclosure describes results showing that TDFRPs prevented apoptosis and inflammation in rat cardiomyocytes, and ameliorated the effects of myocardial ischemia resulting from ischemia/reperfusion injury in vivo. Because the TDFRPs target intracellular pathways that lead to inflammation and apoptosis, the methods described herein are advantageously used to treat or prevent any condition that is associated with myocardial injury

According to one aspect, the disclosure features a method for treating myocardial injury, or a condition associated with myocardial injury, in a mammal, wherein the method comprises administering at least one tissue differentiation factor related polypeptide (TDFRP) to the mammal, and wherein the administering is in an amount effective to treat myocardial injury in said mammal.

According to another aspect, the disclosure features a method for preventing myocardial injury, or a condition associated with myocardial injury, in a mammal, wherein the method comprises administering at least one tissue differentiation factor related polypeptide (TDFRP) to the mammal, and wherein the administering is in an amount effective to prevent myocardial injury in said mammal.

According to some embodiments of the above aspects, the TDFRP is selected from an amino acid sequence at least 90% identical to an amino acid sequence selected from the group consisting of: SEQ ID NO: 1, SEQ ID NO: 2, SEQ ID NO: 3, SEQ ID NO: 4, SEQ ID NO: 5, SEQ ID NO: 6, SEQ ID NO: 7, SEQ ID NO: 8, and SEQ ID NO: 9. According to some embodiments of the above aspects, the TDFRP is selected from an amino acid sequence at least 91%, 92%, 93%, 94%, 95%, 96%, 97%, 98% or 99% identical to an amino acid sequence selected from the group consisting of: SEQ ID NO: 1, SEQ ID NO: 2, SEQ ID NO: 3, SEQ ID NO: 4, SEQ ID NO: 5, SEQ ID NO: 6, SEQ ID NO: 7, SEQ ID NO: 8, and SEQ ID NO: 9. According to some embodiments of the above aspects, the TDFRP comprises SEQ ID NO: 1. According to some embodiments of the above aspects, the TDFRP consists of an amino acid sequence selected from the group consisting of: SEQ ID NO: 1, SEQ ID NO: 2, SEQ ID NO: 3, SEQ ID NO: 4, SEQ ID NO: 5, SEQ ID NO: 6, SEQ ID NO: 7, SEQ ID NO: 8 and SEQ ID NO: 9. According to some embodiments of the above aspects, the TDFRP consists of SEQ ID NO: 1. According to some embodiments of the above aspects, the mammal is a human.

According to some embodiments of the aspects and embodiments herein, the myocardial injury results from or is associated with an ischemia/reperfusion injury. According to some embodiments, the ischemia/reperfusion injury results from myocardial infarction, severe trauma, renal insufficiency, pulmonary insufficiency, arterial stenosis, open heart surgery, or doxorubicin chemotherapy. According to some embodiments of the aspects and embodiments herein, the myocardial injury results from or is associated with a surgical procedure; exposure to a cardiotoxic compound; hypertension; ischemic heart disease; dilated cardiac injury; myocarditis; thyroid disease; viral infection; gingivitis; drug abuse; alcohol abuse; periocarditis; atherosclerosis; vascular disease; hypertrophic cardiomyopathy; acute myocardial infarction; left ventricular systolic dysfunction; coronary bypass surgery; starvation; an eating disorder; or a genetic defect. According to some embodiments, the cardiotoxic compound is an anthracycline, alcohol, or cocaine. According to some embodiments, the anthracyline is doxorubicin, or daunomycin.

According to some embodiments of the aspects and embodiments herein, the TDFRP is administered prior to the diagnosis of the myocardial injury, or the condition associated with myocardial injury in the mammal. According to some embodiments of the aspects and embodiments herein, the TDFRP is administered after the diagnosis of the myocardial injury, or the condition associated with myocardial injury in the mammal. According to some embodiments of the aspects and embodiments herein, the mammal is at risk for myocardial injury. According to some embodiments, the mammal is an individual that smokes, is obese, has been or will be exposed to a cardiotoxic compound, has or had high blood pressure, has or had ischemic heart disease, has or had a myocardial infarct, has a genetic defect that increases the risk of heart failure, has a family history of heart failure, has or had myocardial hypertrophy, has or had hypertrophic cardiomyopathy, has or had left ventricular systolic dysfunction, had coronary bypass surgery, has or had vascular disease, has or had atherosclerosis, has or had alcoholism, has or had pericarditis, has or had a viral infection, has or had gingivitis, has or had an eating disorder, has or had myocarditis, has or had a thyroid disease, or is a cocaine addict.

According to some embodiments of the aspects and embodiments herein, the mammal has or had a cancer.

According to some embodiments of the aspects and embodiments herein, administration of the TDFRP inhibits cardiomyocyte apoptosis.

According to some embodiments of the aspects and embodiments herein, administration of the TDFRP inhibits pericarditis inflammation levels.

According to some embodiments of the aspects and embodiments herein, the TDFRP is administered by a parenteral, intravenous, subcutaneous, intramuscular, intracranial, intraorbital, ophthalmic, intraventricular, intracapsular, intraspinal, intracisternal, intraperitoneal, intranasal, aerosol, oral, or topical route.

According to some embodiments of the aspects and embodiments herein, the method further comprises administration of an additional agent selected from the group consisting of: anti-neoplastic agents, antibiotics, vaccines, immunosuppressive agents, anti-hypertensive agents and mediators of the hedgehog signaling pathway.

### BRIEF DESCRIPTION OF THE DRAWINGS

FIG. 1 is a graph that shows anti-apopototic activity of THR-123 (SEQ ID NO: 1). Phosphorylated Akt is a marker of apoptosis. Ischemic injury leads to apoptosis. As shown in FIG. 1, cell starvation via serum withdrawal led to ischemia and the reduction of phosphorylated AKT in rat cardiomyocytes. Addition of PBS had no effect, whereas addition of THR-123 (SEQ ID NO:1) at 100 μM for 60 h reversed the effects of ischemia on AKT phosphorylation.

FIG. 2 is a graph that shows THR-123 (SEQ ID NO: 1) blocked adriamycin (doxorubicin) suppression of pAKT in human cardiomyocytes, thus demonstrating THR-123 anti-apoptotic activity. As shown in FIG. 2, the addition of 333 nM doxorubicin to serum starved cardiomyocytes further reduces phosphorylation of AKT. Addition of SEQ ID No. 1 at 100 μM and at 500 μM reversed the inhibition of AKT phosphorylation in a dose dependent manner.

FIG. 3 is a graph that shows THR-123 (SEQ ID NO: 1) and BMP-7 bloced adriamycin (doxorubicin) induction of caspase-3 in human cardiomyocytes, thus demonstrating THR-123 anti-apoptotic activity. As shown in FIG. 3, addition of 333 μM doxorubicin to cardiomyocytes stimulated caspase-3 activity. Addition of 143 nM BMP-7 or 100 UM or 500 μM THR-123 (SEQ ID NO: 1) completely reversed the apoptotic effects of doxorubicin.

FIG. 4 is a graph that shows THR-123 (SEQ ID NO: 1) blocked LPS induction of caspase-3 release in human cardiomyocytes, thus demonstrating THR-123 anti-apoptotic activity. As shown in FIG. 4, addition of 100 ng/ml LPS (lipopolysaccharide; an endotoxin) to cardiomyocytes stimulated caspase-3 activity. Addition of 143 nM BMP-7 somewhat reversed the effect of LPS, while 20 μM or higher THR-123 (SEQ ID NO: 1) reversed caspase-3 activity to an even greater extent.

FIG. 5 is a graph that shows BMP-7 and THR-123 (SEQ ID NO: 1) blocked LPS induction of IL-6 in human cardiomyocytes, thus demonstrating anti-inflammatory activity As shown in FIG. 5, exposure of cardiomyocytes to 100 ng/ml LPS resulted in a significant release of inflammatory cytokine IL-6. BMP-7 (143 nM) reduced the level of IL-6 release as did THR-123 in a dose dependent manner.

FIG. 6 shows a schematic of the time course of the Left Anterior Descending Artery (LAD) model.

FIG. 7 shows a schematic of the histomorphometric analysis of the effects of LAD ligation. For each slice of a heart, the area of the necrotic area and the area of the whole area at risk (not stained blue) are measured. The areas are summed over all slices to obtain an estimate of the necrotic volume (NV) and the volume at risk (VR). The ratio of NV/VR is a measure of the myocardial injury resulting from the period of ischemia and subsequent reperfusion injury.

FIG. 8 is a graph showing the results of the histomorphometric analysis. As shown in FIG. 8, there was an 84% reduction in myocardial injury with THR-123 (SEQ ID NO: 1).

FIG. 9 is a graph that shows the effect of treatment on pericarditis on inflammation level.

### DETAILED DESCRIPTION

The present disclosure is based, in part, on the finding that tissue differentiation factor related polypeptides (TDFRPs), and analogs, homologs or variants thereof, can be used to treat or prevent conditions associated with cardiomyopathy, particularly conditions where cardiomyocytes are stressed or injured leading to inflammation, fibrosis, apoptosis, and/or necrosis.

### I. DEFINITIONS

As used in this specification and the appended claims, the singular forms “a”, “an” and “the” include plural references unless the content clearly dictates otherwise.

The use of the alternative (e.g., “or”) should be understood to mean either one, both, or any combination thereof of the alternatives.

As used herein, the term “about,” when referring to a measurable value such as an amount, a temporal duration, and the like, is meant to encompass variations of ±20% or ±10%, more preferably ±5%, even more preferably ±1%, and still more preferably ±0.1% from the specified value, as such variations are appropriate to perform the disclosed methods.

As used herein, any concentration range, percentage range, ratio range, or integer range is to be understood to include the value of any integer within the recited range and, when appropriate, fractions thereof (such as one tenth and one hundredth of an integer), unless otherwise indicated.

As used herein, “comprise,” “comprising,” and “comprises” and “comprised of” are meant to be synonymous with “include”, “including”, “includes” or “contain”, “containing”, “contains” and are inclusive or open-ended terms that specifies the presence of what follows e.g. component and do not exclude or preclude the presence of additional, non-recited components, features, element, members, steps, known in the art or disclosed therein.

As used herein, the terms “such as”, “for example” and the like are intended to refer to exemplary embodiments and not to limit the scope of the present disclosure.

Unless defined otherwise, all technical and scientific terms used herein have the same meaning as commonly understood by one of ordinary skill in the art to which the disclosure pertains. Although any methods and materials similar or equivalent to those described herein can be used in the practice for testing, preferred materials and methods are described herein.

By “acute” is meant a condition having a short course (for example, less than weeks or months), often sudden onset, and resulting from a disease process.

As used herein, “administration,” “administering” and variants thereof refers to introducing a composition or agent into a subject and includes concurrent and sequential introduction of a composition or agent. “Administration” can refer, e.g., to therapeutic, pharmacokinetic, diagnostic, research, placebo, and experimental methods. “Administration” also encompasses in vitro and ex vivo treatments. The introduction of a composition or agent into a subject is by any suitable route, including orally, pulmonarily, intranasally, parenterally (intravenously, intramuscularly, intraperitoneally, or subcutaneously), rectally, intralymphatically, or topically. Administration includes self-administration and the administration by another. Administration can be carried out by any suitable route. A suitable route of administration allows the composition or the agent to perform its intended function. For example, if a suitable route is intravenous, the composition is administered by introducing the composition or agent into a vein of the subject.

As used herein, the term “analog” is meant to refer to a composition that differs from the compound of the present disclosure but retains essential properties thereof. A non-limiting example of this is a polypeptide or peptide or peptide fragment that includes non-natural amino acids, peptidomimetics, unusual amino acids, amide bond isosteres.

As used herein, the term “cardiac remodeling” is meant to refer to a group of molecular, cellular and interstitial changes that manifest clinically as changes in size, mass, geometry and function of the heart after injury. Often, the process results in poor prognosis because of its association with ventricular dysfunction and malignant arrhythmias.

As used herein, the term “cardiomyopathy” is meant to refer to any disease or condition involving injured cardiomyocytes.

As used herein, the term “cardiovascular disease” encompasses diseases and disorders of the muscle and/or blood vessels of the heart, diseases and disorders of the vascular system, and/or diseases and disorders of organs and anatomical systems caused by the diseased condition of the heart and/or vasculature. Examples include, but are not limited to: inflammation of the heart and/or vasculature such as myocarditis, chronic autoimmune myocarditis, bacterial and viral myocarditis, as well as infective endocarditis; heart failure; congestive heart failure; chronic heart failure; cachexia of heart failure; cardiomyopathy, including non-ischemic (dilated cardiomyopathy; idiopathic dilated cardiomyopathy; cardiogenic shock, heart failure secondary to extracorporeal circulatory support (“post-pump syndrome”), heart failure following ischemia/reperfusion injury, brain death associated heart failure (as described in Owen et al., 1999 (Circulation. 1999 May 18; 99 (19): 2565-70)); hypertrophic cardiomyopathy; restrictive cardiomyopathy; non-ischemic systemic hypertension; valvular disease; arythmogenic right ventricular cardiomyopathy) and ischemic (atherogenesis; atherosclerosis; arteriosclerosis; peripheral vascular disease; coronary artery disease; infarctions, including stroke, transient ischemic attacks and myocardial infarctions).

As used herein, the term “myocardial injury” is meant to encompass all conditions causing cardiomyocyte death.

As used herein, the term “conditions associated with myocardial injury” are meant to include any disease or disorder comprising injury of myocardial cells. In some embodiments, conditions associated with myocardial injury are those in which cardiomyocytes are stressed or injured leading to inflammation and fibrosis, apoptosis and/or necrosis.

As used herein the term “myocardial contusion” is a type of myocardial injury and refers to a bruise of the heart muscle. Myocardial contusions may be completely silent or cause an arrhythmia (supraventricular tachycardia or ventricular fibrillation) or hypotension secondary to reduced cardiac output.

As used herein, the term “myocardial fibrosis” is meant to refer to a significant increase in the collagen volume of myocardial tissue. It is a complex process that involves all components of the myocardial tissue and can be triggered by tissue injury from myocardial ischemia (hypoxia), inflammation, and hypertensive overload. To reverse replacement fibrosis, resorption of fibrous tissue needs to be coupled with robust myocardial regeneration. The latter is currently not feasible in adult human hearts. Reversal of interstitial fibrosis poses several challenges (see text) and may require co-operation of several different cell types.

As used herein, the term “congestive heart failure” is meant to refer to a condition of myocardial injury that is characterized by impaired cardiac function that renders the heart unable to maintain the normal blood output at rest or with exercise, or to maintain a normal cardiac output in the setting of normal cardiac filling pressure. A left ventricular ejection fraction of about 40% or less is indicative of congestive heart failure (by way of comparison, an ejection fraction of about 60% percent is normal). Patients in congestive heart failure display well-known clinical symptoms and signs, such as tachypnea, pleural effusions, fatigue at rest or with exercise, contractile dysfunction, and edema. Congestive heart failure is readily diagnosed by well known methods (see, e.g., “Consensus recommendations for the management of chronic heart failure.” Am. J. Cardiol., 83 (2A): 1A-38-A, 1999).

As used herein, the term “ischemic heart disease” is meant to refer to any disorder resulting from an imbalance between the myocardial need for oxygen and the adequacy of the oxygen supply.

Most cases of ischemic heart disease result from narrowing of the coronary arteries, as occurs in atherosclerosis or other vascular disorders.

As used herein, the term “myocardial ischemia” refers to when blood flow to the heart muscle (myocardium) is obstructed by a partial or complete blockage of a coronary artery by a buildup of plaques (atherosclerosis).

As used herein, the term “myocardial infarction” (MI) is meant to refer to a process by which ischemic disease results in a region of the myocardium being replaced by scar tissue.

As used herein, the term “cardiotoxic” is meant a compound that decreases heart function by directing or indirectly impairing or killing cardiomyocytes.

As used herein, the term “hypertension” is meant to refer to blood pressure that is considered by a medical professional (e.g., a physician or a nurse) to be higher than normal and to carry an increased risk for developing congestive heart failure.

As used herein, an “effective amount” of a compound is meant to refer to a quantity sufficient to achieve a desired therapeutic and/or prophylactic effect, for example, an amount which results in the prevention of or a decrease in the symptoms associated with a disease that is being treated, e.g., myocardial injury. The amount of compound administered to the subject will depend on the type and severity of the disease and on the characteristics of the individual, such as general health, age, sex, body weight and tolerance to drugs. It will also depend on the degree, severity and type of disease. The skilled artisan will be able to determine appropriate dosages depending on these and other factors. Typically, an effective amount of the compounds of the present disclosure, sufficient for achieving a therapeutic or prophylactic effect, range from about 0.000001 mg per kilogram body weight per day, to about 10,000 mg per kilogram body weight per day. Preferably, the dosage ranges are from about 0.0001 mg per kilogram body weight per day to about 100 mg per kilogram body weight per day. The compounds of the present disclosure can also be administered in combination with each other, or with one or more additional therapeutic compounds.

As used herein, by “reducing or preventing apoptosis” is meant preventing apoptosis or reducing the levels of apoptosis in a cardiomocyte as compared with an equivalent untreated control; such reduction or degree of prevention is at least 5%, 10%, 20%, 40%, 50%, 60%, 80%, 90%, 95%, or 100% as measured by any standard technique. Standard techniques include for example DNA laddering, TUNEL assay, flow cytometry for DNA content, cell death ELISA, caspase activity, or detection of surrogate markers of apoptosis by immunohistochemistry, Western or Northern analysis.

By “treating, reducing, or preventing cardiac inflammation” is meant preventing inflammation or decreasing the level of inflammation in a heart or cardiac tissue as compared with an equivalent untreated control; such reduction or degree of prevention is at least 5%, 10%, 20%, 40%, 50%, 60%, 80%, 90%, 95%, or 100% as measured by any standard technique. According to this disclosure, inflammation may be measured, for example, by the detection of infiltrating leucocytes (e.g., by immunohistochemistry), the release of pro-inflammatory molecules (e.g., MCP-1), or the activation of inflammatory signaling pathway (e.g., activation of the NF-κB pathway). Thus, the treatment, reduction, or prevention of cardiac inflammation may also be measured by the ability to reduce the activation of inflammatory signaling pathways in a cardiomyocyte as measured by any standard technique.

As used herein, by “reducing or preventing pericarditis” is meant preventing pericarditis or reducing the levels of pericarditis in a cardiomocyte as compared with an equivalent untreated control; such reduction or degree of prevention is at least 5%, 10%, 20%, 40%, 50%, 60%, 80%, 90%, 95%, or 100% as measured by any standard technique.

By “treating, reducing, or preventing ischemic-reperfusion injury” or by “treating, reducing or preventing a cardiac disorder” is meant treating, or ameliorating such injury or cardiac disorder, respectively, before or after it has occurred. As compared with an equivalent untreated control, such reduction or degree of prevention is at least 5%, 10%, 20%, 40%, 50%, 60%, 80%, 90%, 95%, or 100% as measured by any standard technique known in the art.

As used herein, the term “ST elevation” refers to the ST segment of an electrocardiogram where the myocardial cells have gone through depolarization, but not repolarization. This segment should be on the isoelectric line because the there should be no voltage difference across the cardiac muscle cell membrane at this point.

As used herein the term “STEMI” refers to a ST elevation myocardial infarction.

As used herein, the term “NSTEMI” refers to a non-ST elevation myocardial infarction. NSTEMI is a type of heart attack that happens when the heart's need for oxygen cannot be met.

By “iatrogenically-induced” is meant a condition that is of longer duration than acute, and is planned, or is a consequence of a medical treatment (for example, open heart surgery or chemotherapy).

As used herein, an “isolated” or “purified” polypeptide or polypeptide or biologically-active portion thereof is substantially free of cellular material or other contaminating polypeptides from the cell or tissue source from which the tissue differentiation factor-related polypeptide is derived, or substantially free from chemical precursors or other chemicals when chemically synthesized.

As used herein, the terms “polypeptide”, “peptide” and “protein” are used interchangeably herein to refer to a natural or synthetic peptide containing two or more amino acids linked typically via the carboxy group of one amino acid and the amino group of another amino acid. As will be appreciated by those having skill in the art, the above definition is not absolute and polypeptides or peptides can include other examples where one or more amide bonds could be replaced by other bonds, for example, isosteric amide bonds. The terms apply to amino acid polymers in which one or more amino acid residue is an artificial chemical analogue of a corresponding naturally occurring amino acid, as well as to naturally occurring amino acid polymers. The essential nature of such analogues of naturally occurring amino acids is that, when incorporated into a protein, that protein is specifically reactive to antibodies elicited to the same protein but consisting entirely of naturally occurring amino acids. The terms “polypeptide”, “peptide” and “protein” also are inclusive of modifications including, but not limited to, glycosylation, lipid attachment, sulfation, gamma-carboxylation of glutamic acid residues, hydroxylation, and ADP-ribosylation. It will be appreciated, as is well known and as noted above, that polypeptides may not be entirely linear. For instance, polypeptides may be branched as a result of ubiquitination, and they may be circular, with or without branching, generally as a result of posttranslational events, including natural processing event and events brought about by human manipulation which do not occur naturally. Circular, branched and branched circular polypeptides may be synthesized by non-translation natural process and by entirely synthetic methods, as well.

As used herein, the term “pharmaceutically acceptable carrier” includes any of the standard pharmaceutical carriers, such as a phosphate buffered saline solution, water, emulsions such as an oil/water or water/oil, and various types of wetting agents. The term also encompasses any of the agents approved by a regulatory agency of the US Federal government or listed in the US Pharmacopeia for use in animals, including humans, as well as any carrier or diluent that does not cause significant irritation to a subject and does not abrogate the biological activity and properties of the administered compound.

As used herein, the term “small molecule” is meant to refer to a composition that has a molecular weight of less than about 5 kDa and more preferably less than about 2 kDa. Small molecules can be, e.g., nucleic acids, peptides, polypeptides, glycopeptides, peptidomimetics, carbohydrates, lipids, lipopolysaccharides, combinations of these, or other organic or inorganic molecules.

As used herein, the terms “subject,” “individual,” “host,” and “patient,” are used interchangeably herein and refer to any mammalian subject for whom diagnosis, treatment, or therapy is desired, particularly humans. The methods described herein are applicable to both human therapy and veterinary applications. In some embodiments, the subject is a mammal, and in other embodiments the subject is a human. A “subject in need” is meant to refer to a subject that (i) will be administered a TDFRP.

As used herein, the terms “therapeutic amount”, “therapeutically effective amount”, an “amount effective”, or “pharmaceutically effective amount” of an active agent (e.g. a TDFRP), as described herein, are used interchangeably to refer to an amount that is sufficient to provide the intended benefit of treatment. However, dosage levels are based on a variety of factors, including the type of injury, the age, weight, sex, medical condition of the patient, the severity of the condition, the route of administration, and the particular active agent employed. Thus the dosage regimen may vary widely, but can be determined routinely by a physician using standard methods. Additionally, the terms “therapeutic amount”, “therapeutically effective amounts” and “pharmaceutically effective amounts” include prophylactic or preventative amounts of the compositions of the disclosure. In prophylactic or preventative applications of the disclosure, pharmaceutical compositions or medicaments are administered to a patient susceptible to, or otherwise at risk of, a disease, disorder or condition in an amount sufficient to eliminate or reduce the risk, lessen the severity, or delay the onset of the disease, disorder or condition, including biochemical, histologic and/or behavioral symptoms of the disease, disorder or condition, its complications, and intermediate pathological phenotypes presenting during development of the disease, disorder or condition. It is generally preferred that a maximum dose be used, that is, the highest safe dose according to some medical judgment. The terms “dose” and “dosage” are used interchangeably herein.

As used herein, the term “Transforming Growth Factor-beta (TGF-β) superfamily of polypeptides,” is meant to refer to a superfamily of polypeptide factors with pleiotropic functions that is composed of many multifunctional cytokines which includes, but is not limited to, TGF-βs, activins, inhibins, anti-müllerian hormone (AMH), mullerian inhibiting substance (MIS), bone morphogenetic proteins (BMPs), and myostatin. The highly similar TGF-β isoforms TGF-β1, TGF-β2, and TGF-β3 potently inhibit cellular proliferation of many cell types, including those from epithelial origin. Most mesenchymal cells, however, are stimulated in their growth by TGF-β. In addition, TGF-βs strongly induce extracellular matrix synthesis and integrin expression and modulate immune responses. BMPs, also known as osteogenic proteins (OPs), are potent inducers of bone and cartilage formation and play important developmental roles in the induction of ventral mesoderm, differentiation of neural tissue, and organogenesis. Activins, named after their initial identification as activators of follicle-stimulating hormone (FSH) secretion from pituitary glands, are also known to promote erythropoiesis, mediate dorsal mesoderm induction, and contribute to survival of nerve cells. Several growth factors belonging to the TGF-β superfamily play important roles in embryonic patterning and tissue homeostasis. Their inappropriate functioning has been implicated in several pathological situations like fibrosis, rheumatoid arthritis, and carcinogenesis. The term, tissue differentiation factor (TDF), as used herein, includes, but is not limited to, all members of the TGF-beta superfamily of polypeptides. TGF-beta superfamily polypeptides can be antagonists or agonists of TGF-beta superfamily receptors.

As used herein, the term “Transforming Growth Factor-beta (TGF-beta) superfamily receptors,” is meant to refer to polypeptide receptors that mediate the pleiotropic effects of transforming growth factor-β (TGF-β) superfamily polypeptides, as well as fragments, analogs and homologs thereof. Such receptors may include, but are not limited to, distinct combinations of Type I and Type II serine/threonine kinase receptors. The term, tissue differentiation factor receptor (TDF), as used herein, includes, but is not limited to, all members of the TGF-beta superfamily of receptors.

As used herein, “treating” is meant that administration of a TDFRP slows or inhibits the progression of myocardial injury and/or conditions associated with myocardial injury during the treatment, relative to the disease progression that would occur in the absence of treatment, in a statistically significant manner. Well known indicia such as left ventricular ejection fraction, exercise performance, and other clinical tests as enumerated above, as well as survival rates and hospitalization rates may be used to assess disease progression. Whether or not a treatment slows or inhibits disease progression in a statistically significant manner may be determined by methods that are well known in the art (see, e.g., SOLVD Investigators, N. Engl. J. Med. 327:685-691, 1992 and Cohn et al., N. Engl. J. Med. 339:1810-1816, 1998).

As used herein, “preventing” is meant minimizing or partially or completely inhibiting the development of myocardial injury and/or conditions associated with myocardial injury in a mammal at risk for developing congestive heart failure (as defined in “Consensus recommendations for the management of chronic heart failure.” Am. J. Cardiol., 83 (2A): 1A-38-A, 1999). Determination of whether myocardial injury and conditions associated with myocardial injury is minimized or prevented by administration of a TDFRP is made by known methods, such as those described in SOLVD Investigators, supra, and Cohn et al., supra.

According to certain embodiments, being “at risk for myocardial injury” is meant to refer to an individual who smokes, is obese (i.e., 20% or more over their ideal weight), has been or will be exposed to a cardiotoxic compound (such as a chemotherapeutic or an anthracycline antibiotic), or has (or had) high blood pressure, ischemic heart disease, a myocardial infarct, a genetic defect known to increase the risk of heart failure, a family history of heart failure, myocardial hypertrophy, hypertrophic cardiomyopathy, left ventricular systolic dysfunction, coronary bypass surgery, vascular disease, atherosclerosis, alcoholism, periocarditis, a viral infection, gingivitis, or an eating disorder (e.g., anorexia nervosa or bulimia), or is an alcoholic or cocaine addict.

Beneficial or desired clinical results, such as pharmacologic and/or physiologic effects include, but are not limited to, preventing the disease, disorder or condition from occurring in a subject that may be predisposed to the disease, disorder or condition but does not yet experience or exhibit symptoms of the disease (prophylactic treatment), alleviation of symptoms of the disease, disorder or condition, diminishment of extent of the disease, disorder or condition, stabilization (i.e., not worsening) of the disease, disorder or condition, preventing spread of the disease, disorder or condition, delaying or slowing of the disease, disorder or condition progression, amelioration or palliation of the disease, disorder or condition, and combinations thereof, as well as prolonging survival as compared to expected survival if not receiving treatment.

As used herein the term “therapeutic effect” refers to a consequence of treatment, the results of which are judged to be desirable and beneficial. A therapeutic effect can include, directly or indirectly, the arrest, reduction, or elimination of a disease manifestation. A therapeutic effect can also include, directly or indirectly, the arrest reduction or elimination of the progression of a disease manifestation.

For any therapeutic agent described herein the therapeutically effective amount may be initially determined from preliminary in vitro studies and/or animal models. A therapeutically effective dose may also be determined from human data. The applied dose may be adjusted based on the relative bioavailability and potency of the administered compound. Adjusting the dose to achieve maximal efficacy based on the methods described above and other well-known methods is within the capabilities of the ordinarily skilled artisan. General principles for determining therapeutic effectiveness, which may be found in Chapter 1 of Goodman and Gilman's The Pharmacological Basis of Therapeutics, 10th Edition, McGraw-Hill (New York) (2001), incorporated herein by reference, are summarized below.

Pharmacokinetic principles provide a basis for modifying a dosage regimen to obtain a desired degree of therapeutic efficacy with a minimum of unacceptable adverse effects. In situations where the drug's plasma concentration can be measured and related to the therapeutic window, additional guidance for dosage modification can be obtained.

Drug products are considered to be pharmaceutical equivalents if they contain the same active ingredients and are identical in strength or concentration, dosage form, and route of administration. Two pharmaceutically equivalent drug products are considered to be bioequivalent when the rates and extents of bioavailability of the active ingredient in the two products are not significantly different under suitable test conditions.

As used herein, the term “variant,” is meant to refer to a compound that differs from the compound of the present disclosure but retains essential properties thereof. A non-limiting example of this is a polynucleotide or polypeptide compound having conservative substitutions with respect to the reference compound, commonly known as degenerate variants. Another non-limiting example of a variant is a compound that is structurally different but retains the same active domain of the compounds of the present disclosure. Variants include N-terminal or C-terminal extensions, capped amino acids, modifications of reactive amino acid side chain functional groups, e.g., branching from lysine residues, pegylation, and/or truncations of a polypeptide compound. Generally, variants are overall closely similar, and in many regions, identical to the compounds of the present disclosure. Accordingly, the variants may contain alterations in the coding regions, non-coding regions, or both. All technical and scientific terms used herein have the same meaning as commonly understood to one of ordinary skill in the art to which this disclosure belongs unless clearly indicated otherwise.

It is understood that the various embodiments of this disclosure are not limited to the particular methodology, protocols, cell lines, animal species or genera, constructs, and reagents described, as such may vary. It is also understood that the terminology used herein is for the purpose of describing particular embodiments only, and is not intended to limit the scope of the present disclosure which will be limited only by the appended claims.

### II. COMPOSITIONS

According to one aspect, the present disclosure provides compositions comprising at least one TDFRP.

### A. Tissue Differentiation Factor Related Polypeptides (TDFRPs)

The present disclosure provides compounds that are functional analogs of tissue differentiation factors, i.e., compounds that functionally mimic TGF-beta superfamily proteins, for example by acting as TGF-beta superfamily receptor agonists, and preferentially bind to select ALK receptor(s). The present compounds are called TDFRPs, and include small molecules, more particularly polypeptides.

According to one embodiment, the TDFRP compound has the general structure identified as SEQ ID NOs: 1-208, disclosed in International Publication No. WO/2003/106656, incorporated by reference in its entirety herein. According to one embodiment, a TDFRP compound includes an analog or homolog of SEQ ID NOs: 1-208. Compounds of the present disclosure include those with homology to SEQ ID Nos: 1-208, for example, preferably 50% or greater amino acid identity, more preferably 75% or greater amino acid identity, and even more preferably 90% or greater amino acid identity. The compounds of the present disclosure also include one or more polynucleotides encoding SEQ ID Nos: 1-208, including degenerate variants thereof. Accordingly, nucleic acid sequences capable of hybridizing at low stringency with any nucleic acid sequences encoding SEQ ID Nos: 1-208 are considered to be within the scope of the disclosure.

According to one embodiment, the TDFRP compound has the general structure identified as SEQ ID NOs: 1-347, disclosed in International Publication No. WO/2007/035872, incorporated by reference in its entirety herein. According to one embodiment, a TDFRP compound includes an analog or homolog of SEQ ID NOs: 1-347. Compounds of the present disclosure include those with homology to SEQ ID Nos: 1-347, for example, preferably 50% or greater amino acid identity, more preferably 75% or greater amino acid identity, and even more preferably 90% or greater amino acid identity. The compounds of the present disclosure also include one or more polynucleotides encoding SEQ ID Nos: 1-347, including degenerate variants thereof. Accordingly, nucleic acid sequences capable of hybridizing at low stringency with any nucleic acid sequences encoding SEQ ID Nos: 1-347 are considered to be within the scope of the disclosure.

According to one embodiment, the TDFRP compound has the general structure identified as SEQ ID NOs: 1-314, disclosed in International Publication No. WO/2006/009836, incorporated by reference in its entirety herein. According to one embodiment, a TDFRP compound includes an analog or homolog of SEQ ID NOs: 1-314. Compounds of the present disclosure include those with homology to SEQ ID Nos: 1-314, for example, preferably 50% or greater amino acid identity, more preferably 75% or greater amino acid identity, and even more preferably 90% or greater amino acid identity. The compounds of the present disclosure also include one or more polynucleotides encoding SEQ ID Nos: 1-314, including degenerate variants thereof. Accordingly, nucleic acid sequences capable of hybridizing at low stringency with any nucleic acid sequences encoding SEQ ID Nos: 1-314 are considered to be within the scope of the disclosure.

According to one embodiment, the TDFRP compound has the general structure set forth as SEQ ID NOs: 1-77, disclosed in International Publication No. WO/2013/013085 incorporated by reference in its entirety herein. According to one embodiment, a TDFRP compound includes an analog or homolog of SEQ ID NOs: 1-77. Compounds of the present disclosure include those with homology to SEQ ID Nos: 1-77, for example, preferably 50% or greater amino acid identity, more preferably 75% or greater amino acid identity, and even more preferably 90%, 91%, 92%, 93%, 94%, 95%, 96%, 97%, 98% or 99% or greater amino acid identity. The compounds of the present disclosure also include one or more polynucleotides encoding one or more of SEQ ID Nos: 1-77, including degenerate variants thereof. Accordingly, nucleic acid sequences capable of hybridizing at low stringency with any nucleic acid sequences encoding SEQ ID Nos: 1-77 are considered to be within the scope of the disclosure.

Sequence identity can be measured using sequence analysis software (Sequence Analysis Software Package of the Genetics Computer Group, University of Wisconsin Biotechnology Center, 1710 University Avenue, Madison, Wis. 53705), with the default parameters therein.

In the case of polypeptide sequences, which are less than 100% identical to a reference sequence, the non-identical positions are preferably, but not necessarily, conservative substitutions for the reference sequence. Conservative substitutions typically include substitutions within the following groups: glycine and alanine; valine, isoleucine, and leucine; aspartic acid and glutamic acid; asparagine and glutamine; serine and threonine; lysine and arginine; and phenylalanine and tyrosine. Thus, included in the disclosure are peptides having mutated sequences such that they remain homologous, e.g., in sequence, in structure, in function, and in antigenic character or other function, with a polypeptide having the corresponding parent sequence. Such mutations can, for example, be mutations involving conservative amino acid changes, e.g., changes between amino acids of broadly similar molecular properties. For example, interchanges within the aliphatic group alanine, valine, leucine and isoleucine can be considered as conservative. Sometimes substitution of glycine for one of these can also be considered conservative. Other conservative interchanges include those within the aliphatic group aspartate and glutamate; within the amide group asparagine and glutamine; within the hydroxyl group serine and threonine; within the aromatic group phenylalanine, tyrosine, and tryptophan; within the basic group lysine, arginine, and histidine; and within the sulfur-containing group methionine and cysteine. Sometimes substitution within the group methionine and leucine can also be considered conservative. Preferred conservative substitution groups are aspartate-glutamate; asparagine-glutamine; valine-leucine-isoleucine; alanine-valine; phenylalanine-tyrosine; and lysine-arginine.

The disclosure also provides for compounds having altered sequences including insertions such that the overall amino acid sequence is lengthened, while the compound still retains the appropriate TDF agonist or antagonist properties. Additionally, altered sequences may include random or designed internal deletions that truncate the overall amino acid sequence of the compound, however the compound still retains its TDF-like functional properties. According to certain embodiments, one or more amino acid residues within the sequence are replaced with other amino acid residues having physical and/or chemical properties similar to the residues they are replacing. Preferably, conservative amino acid substitutions are those wherein an amino acid is replaced with another amino acid encompassed within the same designated class, as will be described more thoroughly below. Insertions, deletions, and substitutions are appropriate where they do not abrogate the functional properties of the compound. Functionality of the altered compound can be assayed according to the in vitro and in vivo assays described below that are designed to assess the TDF-like properties of the altered compound.

According to some embodiments, particularly preferred peptides include, but are not limited to, the following:

| SEQ ID. No | Seq |
|---|---|
| 10 | CYFDDSSNVLCKKYRS |
| 11 | CYYDNSSSVLCKRYRS |
| 12 | CYYDNSSSVLCKKYRS |
| 13 | CYFDDSSQVICKKYRS |
| 14 | CYFDDSSSVLCKKYRS |
| 15 | CYFDDSSNVLCKKYRN |
| 16 | CYFDDSSNVLCKKYRS |
| 17 | CYYDDSSNVLCKKYRS |
| 18 | CYYDDSSSVLCKKYRS |

According to some embodiments of the embodiments and aspects herein, the peptide is 90%, 91%, 92%, 93%, 94%, 95%, 96%, 97%, 98% or 9% identical to SEQ ID NO: 10. According to some embodiments of the embodiments and aspects herein, the peptide is 90%, 91%, 92%, 93%, 94%, 95%, 96%, 97%, 98% or 9% identical to SEQ ID NO: 11. According to some embodiments of the embodiments and aspects herein, the peptide is 90%, 91%, 92%, 93%, 94%, 95%, 96%, 97%, 98% or 9% identical to SEQ ID NO: 12. According to some embodiments of the embodiments and aspects herein, the peptide is 90%, 91%, 92%, 93%, 94%, 95%, 96%, 97%, 98% or 9% identical to SEQ ID NO: 13. According to some embodiments of the embodiments and aspects herein, the peptide is 90%, 91%, 92%, 93%, 94%, 95%, 96%, 97%, 98% or 9% identical to SEQ ID NO: 14. According to some embodiments of the embodiments and aspects herein, the peptide is 90%, 91%, 92%, 93%, 94%, 95%, 96%, 97%, 98% or 9% identical to SEQ ID NO: 15. According to some embodiments of the embodiments and aspects herein, the peptide is 90%, 91%, 92%, 93%, 94%, 95%, 96%, 97%, 98% or 9% identical to SEQ ID NO: 16. According to some embodiments of the embodiments and aspects herein, the peptide is 90%, 91%, 92%, 93%, 94%, 95%, 96%, 97%, 98% or 9% identical to SEQ ID NO: 17. According to some embodiments of the embodiments and aspects herein, the peptide is 90%, 91%, 92%, 93%, 94%, 95%, 96%, 97%, 98% or 9% identical to SEQ ID NO: 18. According to some embodiments of the embodiments and aspects herein, the peptide consists of SEQ ID NO: 10. According to some embodiments of the embodiments and aspects herein, the peptide consists of SEQ ID NO: 11. According to some embodiments of the embodiments and aspects herein, the peptide consists of SEQ ID NO: 12. According to some embodiments of the embodiments and aspects herein, the peptide consists of SEQ ID NO: 13. According to some embodiments of the embodiments and aspects herein, the peptide consists of SEQ ID NO: 14. According to some embodiments of the embodiments and aspects herein, the peptide consists of SEQ ID NO: 15. According to some embodiments of the embodiments and aspects herein, the peptide consists of SEQ ID NO: 16. According to some embodiments of the embodiments and aspects herein, the peptide consists of SEQ ID NO: 17. According to some embodiments of the embodiments and aspects herein, the peptide consists of SEQ ID NO: 18.

According to some embodiments, SEQ ID NOs 10-18 set forth above further comprise a N-terminal (H) and a C-terminal (OH):

| SEQ ID. No | NT | Seq | CT |
|---|---|---|---|
| 1 | (H) | CYFDDSSNVLCKKYRS | (OH) |
| 2 | (H) | CYYDNSSSVLCKRYRS | (OH) |
| 3 | (H) | CYYDNSSSVLCKKYRS | (OH) |
| 4 | (H) | CYFDDSSQVICKKYRS | (OH) |
| 5 | (H) | CYFDDSSSVLCKKYRS | (OH) |
| 6 | (H) | CYFDDSSNVLCKKYRN | (OH) |
| 7 | (H) | CYFDDSSNVLCKKYRS | (NH2) |
| 8 | (H) | CYYDDSSNVLCKKYRS | (OH) |
| 9 | (H) | CYYDDSSSVLCKKYRS | (OH) |

According to some embodiments of the embodiments and aspects herein, the peptide is 90%, 91%, 92%, 93%, 94%, 95%, 96%, 97%, 98% or 9% identical to SEQ ID NO: 1. According to some embodiments of the embodiments and aspects herein, the peptide is 90%, 91%, 92%, 93%, 94%, 95%, 96%, 97%, 98% or 9% identical to SEQ ID NO: 2. According to some embodiments of the embodiments and aspects herein, the peptide is 90%, 91%, 92%, 93%, 94%, 95%, 96%, 97%, 98% or 9% identical to SEQ ID NO: 3. According to some embodiments of the embodiments and aspects herein, the peptide is 90%, 91%, 92%, 93%, 94%, 95%, 96%, 97%, 98% or 9% identical to SEQ ID NO: 4. According to some embodiments of the embodiments and aspects herein, the peptide is 90%, 91%, 92%, 93%, 94%, 95%, 96%, 97%, 98% or 9% identical to SEQ ID NO: 5. According to some embodiments of the embodiments and aspects herein, the peptide is 90%, 91%, 92%, 93%, 94%, 95%, 96%, 97%, 98% or 9% identical to SEQ ID NO: 6. According to some embodiments of the embodiments and aspects herein, the peptide is 90%, 91%, 92%, 93%, 94%, 95%, 96%, 97%, 98% or 9% identical to SEQ ID NO: 7. According to some embodiments of the embodiments and aspects herein, the peptide is 90%, 91%, 92%, 93%, 94%, 95%, 96%, 97%, 98% or 9% identical to SEQ ID NO: 8. According to some embodiments of the embodiments and aspects herein, the peptide is 90%, 91%, 92%, 93%, 94%, 95%, 96%, 97%, 98% or 9% identical to SEQ ID NO: 9. According to some embodiments of the embodiments and aspects herein, the peptide consists of SEQ ID NO: 1. According to some embodiments of the embodiments and aspects herein, the peptide consists of SEQ ID NO: 2. According to some embodiments of the embodiments and aspects herein, the peptide consists of SEQ ID NO: 3. According to some embodiments of the embodiments and aspects herein, the peptide consists of SEQ ID NO: 4. According to some embodiments of the embodiments and aspects herein, the peptide consists of SEQ ID NO: 5. According to some embodiments of the embodiments and aspects herein, the peptide consists of SEQ ID NO: 6. According to some embodiments of the embodiments and aspects herein, the peptide consists of SEQ ID NO: 7. According to some embodiments of the embodiments and aspects herein, the peptide consists of SEQ ID NO: 8. According to some embodiments of the embodiments and aspects herein, the peptide consists of SEQ ID NO: 9.

### (i) TDFRP Recombinant Expression Vectors

According to one aspect, the disclosure includes vectors containing one or more nucleic acid sequences encoding a TDFRP compound. For recombinant expression of one or more the polypeptides of the disclosure, the nucleic acid containing all or a portion of the nucleotide sequence encoding the polypeptide is inserted into an appropriate cloning vector, or an expression vector (i.e., a vector that contains the necessary elements for the transcription and translation of the inserted polypeptide coding sequence) by recombinant DNA techniques well known in the art and as detailed below.

In general, expression vectors useful in recombinant DNA techniques are often in the form of plasmids. In the present specification, “plasmid” and “vector” can be used interchangeably as the plasmid is the most commonly used form of vector. However, the disclosure is intended to include such other forms of expression vectors that are not technically plasmids, such as viral vectors (e.g., replication defective retroviruses, adenoviruses and adeno-associated viruses), which serve equivalent functions. Such viral vectors permit infection of a subject and expression in that subject of a compound.

The recombinant expression vectors of the disclosure comprise a nucleic acid encoding a compound with TDF-like properties in a form suitable for expression of the nucleic acid in a host cell, which means that the recombinant expression vectors include one or more regulatory sequences, selected on the basis of the host cells to be used for expression that is operatively-linked to the nucleic acid sequence to be expressed. Within a recombinant expression vector, “operably-linked” is intended to mean that the nucleotide sequence of interest is linked to the regulatory sequence(s) in a manner that allows for expression of the nucleotide sequence (e.g., in an in vitro transcription/translation system or in a host cell when the vector is introduced into the host cell).

The term “regulatory sequence” is intended to include promoters, enhancers and other expression control elements (e.g., polyadenylation signals). Such regulatory sequences are described, for example, in Goeddel, GENE EXPRESSION TECHNOLOGY: METHODS IN ENZYMOLOGY 185, Academic Press, San Diego, Calif. (1990). Regulatory sequences include those that direct constitutive expression of a nucleotide sequence in many types of host cell and those that direct expression of the nucleotide sequence only in certain host cells (e.g., tissue-specific regulatory sequences). It will be appreciated by those skilled in the art that the design of the expression vector can depend on such factors as the choice of the host cell to be transformed, the level of expression of polypeptide desired, etc. The expression vectors of the disclosure can be introduced into host cells to thereby produce polypeptides or peptides, including fusion polypeptides, encoded by nucleic acids as described herein (e.g., TDFRP compounds and TDFRP-derived fusion polypeptides, etc.).

### (ii) TDFRP-Expressing Host Cells

According to another aspect, the present disclosure pertains to TDFRP-expressing host cells, which contain a nucleic acid encoding one or more TDFRP compounds. The recombinant expression vectors of the disclosure can be designed for expression of TDFRP compounds in prokaryotic or eukaryotic cells. For example, TDFRP compounds can be expressed in bacterial cells such as Escherichia coli, insect cells (using baculovirus expression vectors), fungal cells, e.g., yeast, yeast cells or mammalian cells. Suitable host cells are discussed further in Goeddel, GENE EXPRESSION TECHNOLOGY: METHODS IN ENZYMOLOGY 185, Academic Press, San Diego, CA. (1990). Alternatively, the recombinant expression vector can be transcribed and translated in vitro, for example using T7 promoter regulatory sequences and T7 polymerase.

Expression of polypeptides in prokaryotes is most often carried out in Escherichia coli with vectors containing constitutive or inducible promoters directing the expression of either fusion or non-fusion polypeptides. Fusion vectors add a number of amino acids to a polypeptide encoded therein, usually to the amino terminus of the recombinant polypeptide. Such fusion vectors typically serve three purposes: (i) to increase expression of recombinant polypeptide; (ii) to increase the solubility of the recombinant polypeptide; and (iii) to aid in the purification of the recombinant polypeptide by acting as a ligand in affinity purification. Often, in fusion expression vectors, a proteolytic cleavage site is introduced at the junction of the fusion moiety and the recombinant polypeptide to enable separation of the recombinant polypeptide from the fusion moiety subsequent to purification of the fusion polypeptide. Such enzymes, and their cognate recognition sequences, include Factor Xa, thrombin and enterokinase. Typical fusion expression vectors include pGEX (Pharmacia Biotech Inc; Smith and Johnson, 1988. Gene 67:31-40), pMAL (New England Biolabs, Beverly, Mass.) and pRIT5 (Pharmacia, Piscataway, N.J.) that fuse glutathione S-transferase (GST), maltose E binding polypeptide, or polypeptide A, respectively, to the target recombinant polypeptide.

Examples of suitable inducible non-fusion E. coli expression vectors include pTrc (Amrann et al., (1988) Gene 69:301-315) and pET11d (Studier et al., GENE EXPRESSION TECHNOLOGY: METHODS IN ENZYMOLOGY 185, Academic Press, San Diego, Calif. (1990) 60-89).

One strategy to maximize recombinant polypeptide expression in E. coli is to express the polypeptide in host bacteria with an impaired capacity to proteolytically cleave the recombinant polypeptide. See, e.g., Gottesman, GENE EXPRESSION TECHNOLOGY: METHODS IN ENZYMOLOGY 185, Academic Press, San Diego, CA. (1990) 119-128. Another strategy is to alter the nucleic acid sequence of the nucleic acid to be inserted into an expression vector so that the individual codons for each amino acid are those preferentially utilized in the expression host, e.g., E. coli (see, e.g., Wada, et al., 1992. Nucl. Acids Res. 20:2111-2118). Such alteration of nucleic acid sequences of the disclosure can be carried out by standard DNA synthesis techniques.

In another embodiment, the TDFRP expression vector is a yeast expression vector. Examples of vectors for expression in yeast Saccharomyces cerivisae include pYepSec1 (Baldari, et al., 1987. EMBO J. 6:229-234), pMFa (Kurjan and Herskowitz, 1982. Cell 30:933-943), pJRY88 (Schultz et al., 1987. Gene 54:113-123), pYES2 (Invitrogen Corporation, San Diego, CA.), and picZ (Invitrogen Corp, San Diego, CA.). Alternatively, TDFRP can be expressed in insect cells using baculovirus expression vectors. Baculovirus vectors available for expression of polypeptides in cultured insect cells (e.g., SF9 cells) include the pAc series (Smith, et al., 1983. Mol. Cell. Biol. 3:2156-2165) and the pVL series (Lucklow and Summers, 1989. Virology 170:31-39).

In yet another embodiment, a nucleic acid of the disclosure is expressed in mammalian cells using a mammalian expression vector. Examples of mammalian expression vectors include pCDM8 (Seed, 1987. Nature 329:840) and pMT2PC (Kaufman, et al., 1987. EMBO J. 6:187-195). When used in mammalian cells, the expression vector's control functions are often provided by viral regulatory elements. For example, commonly used promoters are derived from polyoma, adenovirus 2, cytomegalovirus, and simian virus 40. For other suitable expression systems for both prokaryotic and eukaryotic cells see, e.g., Chapters 16 and 17 of Sambrook, et al., MOLECULAR CLONING: A LABORATORY MANUAL. 2^{nd} ed., Cold Spring Harbor Laboratory, Cold Spring Harbor Laboratory Press, Cold Spring Harbor, NY, 1989.

In another embodiment, the recombinant mammalian expression vector is capable of directing expression of the nucleic acid preferentially in a particular cell type (e.g., tissue-specific regulatory elements are used to express the nucleic acid). Tissue-specific regulatory elements are known in the art. Non-limiting examples of suitable tissue-specific promoters include the albumin promoter (liver-specific; Pinkert, et al., 1987. Genes Dev. 1:268-277), lymphoid-specific promoters (Calame and Eaton, 1988. Adv. Immunol. 43:235-275), in particular promoters of T cell receptors (Winoto and Baltimore, 1989. EMBO J. 8:729-733) and immunoglobulins (Banerji, et al., 1983. Cell 33:729-740; Queen and Baltimore, 1983. Cell 33:741-748), neuron-specific promoters (e.g., the neurofilament promoter; Byrne and Ruddle, 1989. Proc. Natl. Acad. Sci. USA 86:5473-5477), pancreas-specific promoters (Edlund, et al., 1985. Science 230:912-916), and mammary gland-specific promoters (e.g., milk whey promoter; U.S. Pat. No. 4,873,316 and European Application Publication No. 264,166). Developmentally-regulated promoters are also encompassed, e.g., the murine hox promoters (Kessel and Gruss, 1990. Science 249:374-379) and the α-fetoprotein promoter (Campes and Tilghman, 1989. Genes Dev. 3:537-546).

The disclosure further provides a recombinant expression vector comprising a DNA molecule of the disclosure cloned into the expression vector in an antisense orientation. That is, the DNA molecule is operatively linked to a regulatory sequence in a manner that allows for expression (by transcription of the DNA molecule) of an RNA molecule that is antisense to a TDRFP mRNA. Regulatory sequences operatively linked to a nucleic acid cloned in the antisense orientation can be chosen that direct the continuous expression of the antisense RNA molecule in a variety of cell types, for instance viral promoters and/or enhancers, or regulatory sequences can be chosen that direct constitutive, tissue specific or cell type specific expression of antisense RNA. The antisense expression vector can be in the form of a recombinant plasmid, phagemid or attenuated virus in which antisense nucleic acids are produced under the control of a high efficiency regulatory region, the activity of which can be determined by the cell type into which the vector is introduced. For a discussion of the regulation of gene expression using antisense genes see, e.g., Weintraub, et al., “Antisense RNA as a molecular tool for genetic analysis,” Reviews-Trends in Genetics, Vol. 1 (1) 1986.

Another aspect of the disclosure pertains to host cells into which a recombinant expression vector of the disclosure has been introduced. The terms “host cell” and “recombinant host cell” are used interchangeably herein. It is understood that such terms refer not only to the particular subject cell but also to the progeny or potential progeny of such a cell. Because certain modifications may occur in succeeding generations due to either mutation or environmental influences, such progeny may not, in fact, be identical to the parent cell, but are still included within the scope of the term as used herein.

A host cell can be any prokaryotic or eukaryotic cell. For example, TDFRP can be expressed in bacterial cells such as E. coli, insect cells, yeast or mammalian cells (such as Chinese hamster ovary cells (CHO) or COS cells). Other suitable host cells are known to those skilled in the art.

Vector DNA can be introduced into prokaryotic or eukaryotic cells via conventional transformation or transfection techniques. As used herein, the terms “transformation” and “transfection” are intended to refer to a variety of art-recognized techniques for introducing foreign nucleic acid (e.g., DNA) into a host cell, including calcium phosphate or calcium chloride co-precipitation, DEAE-dextran-mediated transfection, lipofection, or electroporation. Suitable methods for transforming or transfecting host cells can be found in Sambrook, et al. (MOLECULAR CLONING: A LABORATORY MANUAL. 2^{nd} ed., Cold Spring Harbor Laboratory, Cold Spring Harbor Laboratory Press, Cold Spring Harbor, N.Y., 1989), and other laboratory manuals.

For stable transfection of mammalian cells, it is known that, depending upon the expression vector and transfection technique used, only a small fraction of cells may integrate the foreign DNA into their genome. In order to identify and select these integrants, a gene that encodes a selectable marker (e.g., resistance to antibiotics) is generally introduced into the host cells along with the gene of interest. Various selectable markers include those that confer resistance to drugs, such as G418, hygromycin and methotrexate. Nucleic acid encoding a selectable marker can be introduced into a host cell on the same vector as that encoding TDFRP or can be introduced on a separate vector. Cells stably transfected with the introduced nucleic acid can be identified by drug selection (e.g., cells that have incorporated the selectable marker gene will survive, while the other cells die).

A host cell that includes a compound of the disclosure, such as a prokaryotic or eukaryotic host cell in culture can be used to produce (i.e., express) recombinant TDFRP. According to one embodiment, the method comprises culturing the host cell of disclosure (into which a recombinant expression vector encoding TDFRP has been introduced) in a suitable medium such that TDFRP is produced. According to another embodiment, the method further comprises the step of isolating TDFRP from the medium or the host cell. Purification of recombinant polypeptides is well-known in the art and include ion-exchange purification techniques, or affinity purification techniques, for example with an antibody to the compound.

(iii) TDFRP-Derived Chimeric and Fusion Polypeptides

The present disclosure also provides for compounds that are TDFRP-derived chimeric or fusion polypeptides. As used herein, a TDFRP-derived “chimeric polypeptide” or “fusion polypeptide” comprises a TDFRP operatively-linked to a polypeptide having an amino acid sequence corresponding to a polypeptide that is not substantially homologous to the TDFRP, e.g., a polypeptide that is different from the TDFRP and that is derived from the same or a different organism (i.e., non-TDFRP). Within a TDFRP-derived fusion polypeptide, the TDFRP can correspond to all or a portion of a TDFRP. According to one embodiment, a TDFRP-derived fusion polypeptide comprises at least one biologically-active portion of a TDFRP, for example a fragment of SEQ ID Nos: 1-347. According to another embodiment, a TDFRP-derived fusion polypeptide comprises at least two biologically active portions of a TDFRP. In yet another embodiment, a TDFRP-derived fusion polypeptide comprises at least three biologically active portions of a TDFRP polypeptide. Within the fusion polypeptide, the term “operatively linked” is intended to indicate that the TDFRP polypeptide and the non-TDFRP polypeptide are fused in-frame with one another. The non-TDFRP polypeptide can be fused to the N-terminus or C-terminus of the TDFRP.

According to one embodiment, the fusion polypeptide is a GST-TDFRP fusion polypeptide in which the TDFRP sequences are fused to the N- or C-terminus of the GST (glutathione S-transferase) sequences. Such fusion polypeptides can facilitate the purification of recombinant TDFRP by affinity means.

According to another embodiment, the fusion polypeptide is a TDFRP polypeptide containing a heterologous signal sequence at its N-terminus. In certain host cells (e.g., mammalian host cells), expression and/or secretion of TDFRP can be increased through use of a heterologous signal sequence.

According to yet another embodiment, the fusion polypeptide is a TDFRP-immunoglobulin fusion polypeptide in which the TDFRP sequences are fused to sequences derived from a member of the immunoglobulin superfamily. The TDFRP-immunoglobulin fusion polypeptides of the disclosure can be incorporated into pharmaceutical compositions and administered to a subject to inhibit an interaction between a TDF and a TDF receptor polypeptide on the surface of a cell, to thereby suppress TDF-mediated signal transduction in vivo. The TDFRP-immunoglobulin fusion polypeptides can be used to affect the bioavailability of a TDFRP, for example to target the compound to a particular cell or tissue having the requisite antigen. Inhibition of the TDF/TDF receptor interaction can be useful therapeutically for both the treatment of proliferative and differentiative disorders, as well as modulating (e.g., promoting or inhibiting) cell survival.

### (iv) TDFRP1-Linker-TDFRP2

The TDFRP compounds described herein contain multiple TDF-related polypeptides (i.e., multiple domain TDF-related polypeptide compounds, hereinafter “TDFRP”) with the general structure shown below:

### TDFRP1-Linker-TDFRP2

Where a first TDFRP domain (TDFRP1, i.e., TDF-related polypeptide 1) is covalently linked via the C-terminus, N-terminus, or any position with a functionalizable side group, e.g., lysine or aspartic acid to a linker molecule, which, in turn, is covalently linked to the N-terminus of a second TDFRP domain (TDFRP2).

The TDRFP domains are compounds that include small molecules. Variants, analogs, homologs, or fragments of these compounds, such as species homologs, are also included in the present disclosure, as well as degenerate forms thereof.

A first domain is linked to a second domain through a linker. The term “linker,” as used herein, refers to an element capable of providing appropriate spacing or structural rigidity, or structural orientation, alone, or in combination, to a first and a second domain, e.g., TDFRP1 and TDFRP2, such that the biological activity of the TDFRP is preserved. For example, linkers may include, but are not limited to, a diamino alkane, a dicarboxylic acid, an amino carboxylic acid alkane, an amino acid sequence, e.g., glycine polypeptide, a disulfide linkage, a helical or sheet-like structural element or an alkyl chain. According to one aspect the linker is not inert, e.g., chemically or enzymatically cleavable in vivo or in vitro. In another aspect the linker is inert, i.e., substantially unreactive in vivo or in vitro, e.g., is not chemically or enzymatically degraded. Examples of inert groups which can serve as linking groups include aliphatic chains such as alkyl, alkenyl and alkynyl groups (e.g., C1-C20), cycloalkyl rings (e.g., C3-C10), aryl groups (carbocyclic aryl groups such as 1-naphthyl, 2-naphthyl, 1-anthracyl and 2-anthracyl and heteroaryl group such as N-imidazolyl, 2-imidazole, 2-thienyl, 3-thienyl, 2-furanyl, 3-furanyl, 2-pyridyl, 3-pyridyl, 4-pyridyl, 2-pyrimidy, 4-pyrimidyl, 2-pyranyl, 3-pyranyl, 3-pyrazolyl, 4-pyrazolyl, 5-pyrazolyl, 2-pyrazinyl, 2-thiazole, 4-thiazole, 5-thiazole, 2-oxazolyl, 4-oxazolyl, 5-oxazolyl, 2-benzothienyl, 3-benzothienyl, 2-benzofuranyl, 3-benzofuranyl, 2-indolyl, 3-indolyl, 2-quinolinyl, 3-quinolinyl, 2-benzothiazole, 2-benzooxazole, 2-benzimidazole, 2-quinolinyl, 3-quinolinyl, 1-isoquinolinyl, 3-quinolinyl, 1-isoindolyl, and 3-isoindolyl), non-aromatic heterocyclic groups (e.g., 2-tetrahydrofuranyl, 3-tetrahydrofuranyl, 2-tetrahyrothiophenyl, 3-tetrahyrothiophenyl, 2-morpholino, 3-morpholino, 4-morpholino, 2-thiomorpholino, 3-thiomorpholino, 4-thiomorpholino, 1-pyrrolidinyl, 2-pyrrolidinyl, 3-pyrrolidinyl, 1-piperazinyl, 2-piperazinyl, 1-piperidinyl, 2-piperidinyl, 3-piperidinyl, 4-piperidinyl and 4-thiazolidinyl) and aliphatic groups in which one, two or three methylenes have been replaced with —O—, —S—, —NH—, —SO₂—, —SO— or —SO₂NH—.

The TDFRP compounds include small molecules, more particularly TDFRP compound domains, with the general structure identified herein, as detailed below. The TDFRP compound domains disclosed herein may be present in an TDFRP compound in any combination or orientation. Variants, analogs, homologs, or fragments of these TDFRP compound domains, such as species homologs, are also included in the present disclosure, as well as degenerate forms thereof. The TDFRP compound domains of the present disclosure may be capped on the N-terminus, or the C-terminus, or on both the N-terminus and the C-terminus. The TDFRP compounds may be pegylated, or modified, e.g., branching, at any amino acid residue containing a reactive side chain, e.g., lysine residue, or chemically reactive group on the linker. The TDFRP compound of the present disclosure may be linear or cyclized. The tail sequence of the TDFRP or TDFRP domains may vary in length. According to one aspect of the present disclosure, the TDFRP compounds of the disclosure are prodrugs, i.e., the biological activity of the TDFRP compound is altered, e.g., increased, upon contacting a biological system in vivo or in vitro.

The TDFRP compounds can contain natural amino acids, non-natural amino acids, d-amino acids and l-amino acids, and any combinations thereof. According to certain embodiments, the compounds of the disclosure can include commonly encountered amino acids, which are not genetically encoded. These non-genetically encoded amino acids include, but are not limited to, β-alanine (β-Ala) and other omega-amino acids such as 3-aminopropionic acid (Dap), 2,3-diaminopropionic acid (Dpr), 4-aminobutyric acid and so forth; α-aminoisobutyric acid (Aib); ε-aminohexanoic acid (Aha); 8-aminovaleric acid (Ava); N-methylglycine or sarcosine (MeGly); ornithine (Orn); citrulline (Cit); t-butylalanine (t-BuA); t-butylglycine (t-BuG); N-methylisoleucine (Melle); phenylglycine (Phg); cyclohexylalanine (Cha); norleucine (Nle); 2-naphthylalanine (2-Nal); 4-chlorophenylalanine (Phe (4-Cl)); 2-fluorophenylalanine (Phe (2-F)); 3-fluorophenylalanine (Phe (3-F)); 4-fluorophenylalanine (Phe (4-F)); penicillamine (Pen); 1,2,3,4-tetrahydroisoquinoline-3-carboxylic acid (Tic); β-2-thienylalanine (Thi); methionine sulfoxide (MSO); homoarginine (hArg); N-acetyl lysine (AcLys); 2,3-diaminobutyric acid (Dab); 2,3-diaminobutyric acid (Dbu); p-aminophenylalanine (Phe (pNH2)); N-methyl valine (MeVal); homocysteine (hCys) and homoserine (hSer). Non-naturally occurring variants of the compounds may be produced by mutagenesis techniques or by direct synthesis.

### B. Measurement of TDFRP Biological Activity

The biological activity, namely the agonist or antagonist properties of TDF polypeptides or TDFRP compounds can be characterized using any conventional in vivo and in vitro assays that have been developed to measure the biological activity of the TDFRP compound, a TDF polypeptide or a TDF signaling pathway component.

TGF-b/BMPs Superfamily members are associated with a number of cellular activities involved in injury responses and regeneration. TDFRP compounds can be used as agonists of BMPs or antagonists of TGF-b molecules to mediate activities that can prevent, repair or alleviate injurious responses in cells, tissues or organs. Key activities involved in mediating these effects would be anti-inflammatory, anti-apoptotic and anti-fibrotic properties. Several in vitro models for inflammation can be used to assess cytokine, chemokine and cell adhesion responses, which are well-documented markers of inflammation. Cellular injury induced by tumor necrosis factor-alpha (TNF-α), cisplatin, lipopolysaccharide (LPS) or other agents lead to the production of pro-inflammatory molecules (for example, IL-1, IL-6, IL-8, NF-kappaB) and adhesion molecules (for example, intercellular adhesion molecule-1 or ICAM-1). Furthermore, these agents induce chemokines (IL-6, IL-8, monocyte chemoattractant protein-1 or MCP-1 and RANTES), which cause immune cells to infiltrate tissues resulting in organ damage.

Apoptosis or programmed cell death is initiated through either a mitochondrial pathway, in response to stress factors or through a receptor-mediated pathway, triggered by the binding of ligands, such as TNF-α. Multiple factors contribute to the complex apoptotic process, including the infiltration neutrophils and other inflammatory cells that activate a class of enzymes known as caspases. Other useful markers of apoptosis are Bax and the human vascular anticoagulant, Annexin V, which binds to a protein that gets translocated from the inner to the outer plasma membrane in apoptotic cells.

In a typical experiment, the anti-apoptotic activity of TDFRP compounds can be assessed using in vitro models of apoptosis in cultured cells (for example, heart muscle cells or cardiomyocytes).

Cardiomyocytes rarely proliferate in adult cardiac muscles and the loss of cardiac muscle cells can lead to permanent loss of cardiac function. Myocardial apoptosis, caused by injury to cardiomyoctes, contributes to or aggravates the development of myocardial dysfunction in various cardiac diseases. The chemotherapeutic agent, doxorubicin causes heart failure, a reduced number of functioning cardiac muscle cells, activation of caspase 3 and apoptosis. In a typical experiment, the anti-apoptotic activity of TDFRP compounds can be demonstrated by showing the inhibition of Bax and caspase-3 expression that was induced by doxorubicin, LPS or ischemia, as well as by showing an increase in the levels of phosphorylated Akt, a sensitive indicator of cardiomyocyte health.

Myocardial injury (MI) arises when a decrease in blood flow or obstruction in one of the major arteries feeding the heart muscle prevents oxygen and nutrients from reaching the heart muscle. The first phase of this process is an ischemic state, where decreased blood flow restricts the oxygen supply below the demand required by the heart muscle. The myocardial cells are starved for the oxygen and nutrients carried by the blood. This situation can only be sustained for very short periods of time before the cells become irreversibly injured and die. The death of these cells constitutes an infarction, and the injured cells release chemicals that incite inflammatory reactions.

Myocardial ischemia (MI) leads to apoptosis, inflammation, and fibrosis. TPA, administered soon after a myocardial infarction, limits damage (by reopening the blood vessel) but has no effect on the cellular processes of apoptosis, inflammation, and fibrosis.

In a typical experiment, TDFRP compounds can be used to demonstrate anti-inflammatory and anti-apoptotic activities in cultured cardiomyoctes. In a typical animal model study, TDFRP compounds can be used to reduce the size of infarct, maintain coronary artery endothelial function and inhibit neutrophil adherence to vascular endothelium (reduced reperfusion injury) in rat models of MI. The MI rat model (called the Left Anterior Descending Artery (LAD) occlusion model) involves the transient ligation of the left anterior descending artery to create ischemia. Upon removal of the ligature, blood flow into the heart initiates reperfusion injury, which can be monitored by perfusing the area with dye and assessing the degree of infarct. In a typical LAD experiment, TDFRP compounds can be administered before and after ischemia induced by ligation of the heart ventricle. Efficacy can be determined by morphology and by assessing Creatine Kinase-Myocardioband (CK-MB) levels between infarct and non-infarct regions of the ventricle following reperfusion.

### III. METHODS

Provided herein are methods of treatment and prevention using TDFRPs to block and reverse the intracellular pathways in cardiomyocytes that lead to inflammation and apoptosis, thereby preventing necrosis and fibrosis.

According to one aspect, the disclosure features a method of treating a condition that is associated with myocardial injury, the method comprising administering to a subject in need of treatment at least one tissue differentiation factor related polypeptide (TDFRP). According to another aspect, the disclosure features a method of preventing a condition that is associated with myocardial injury, the method comprising administering to a subject in need of prevention at least one TDFRP.

There are a number of ways in which cardiomyocytes can be injured. Some of those injuries are immediate, and include, but are not limited to: ischemia, myocardial infarction, chronic arterial insufficiency, atherosclerosis, mechanical stress (e.g., over work of cardiomyocytes), pressure overload, due to valvular obstruction, hypertension, volume overload and arrhythmias.

Various clinical disorders can also lead to myocardial injury. Congenital disorders include Coarctation, where section of the aorta developed narrow; atrial septal defect, a hole between two upper chambers of the heart; ventricular septal defect, a hole between two lower chambers of the heart; noncompaction, a developmental defect in which the lower left ventricle failed to develop; and pulmonary hypertension, narrowed arteries in lung and heart.

Infectious diseases can also lead to myocardial injury, and include, but are not limited to, viral, bacterial, fungal infections and Ricksttsia.

Autoimmune diseases can also lead to myocardial injury, and include, but are not limited to, Systemic Lupus Erythematosis and Scleroderma. In both of these cases, TDFRPs represent a possible chronic treatment or preventative treatment for myocardial injury associated with the autoimmune diseases.

Cardiotoxic compound (e.g., cocaine, alcohol, an anti-ErbB2 antibody or anti-HER2 antibody, such as HERCEPTIN, or an anthracycline antibiotic, such as doxorubicin or daunomycin) can lead to myocardial injury. TDFRPs represent a possible chronic treatment or preventative treatment for myocardial injury associated with cardiotoxic compounds. For example, TDFRP administration to cancer patients prior to and during anthracycline chemotherapy or anthracycline/anti-ErbB2 (anti-HER2) antibody (e.g., HERCEPTIN) combination therapy may prevent the patients' cardiomyocytes from undergoing apoptosis, thereby preserving cardiac function.

Some genetic defects are known to increase the risk of heart failure (such as those described in Bachinski and Roberts, Cardiol. Clin. 16:603-610, 1998; Siu et al., Circulation 8:1022-1026, 1999; and Arbustini et al., Heart 80:548-558, 1998).

Other various factors and conditions that can lead to myocardial injury include, but are not limited to, toxins, heavy metals, doxorubicin, gingivitis, drug abuse, alcohol abuse, starvation, amphetamines, cancer treatments, genetic or congenital conditions, missing, unusual, or abnormal protein expression (extracellular-interacting with cardiomyocytes or intracellular-internal structural or signaling).

Any of the above conditions may be treated or prevented using the methods described herein.

### A. Cardiac Injury (CI)

Cardiac injury refers to a group of diseases that affect the heart muscle. Early on there may be few or no symptoms of cardiac injury. However, as the disease worsens, shortness of breath, feeling tired, and swelling of the legs may occur, due to the onset of heart failure. An irregular heart beat and fainting may occur. Those affected are at an increased risk of sudden cardiac death.

Types of cardiac injury include: ischemic cardiomyopathy, hypertrophic cardiomyopathy, dilated cardiomyopathy, restrictive cardiomyopathy, arrhythmogenic right ventricular dysplasia, and Takotsubo cardiomyopathy (broken heart syndrome) (CI-3). In hypertrophic cardiomyopathy the heart muscle enlarges and thickens. In dilated cardiomyopathy the ventricles enlarge and weaken. In restrictive cardiomyopathy the ventricle stiffens.

### (i) Hypertrophic Cardiomyopathy (HC)

Hypertrophic cardiomyopathy (HCM, or HOCM when obstructive) is a condition in which the heart becomes thickened without an obvious cause. The parts of the heart most commonly affected are the interventricular septum and the ventricles. This results in the heart being less able to pump blood effectively and also may cause electrical conduction problems. In hypertrophic cardiomyopathy the heart muscle enlarges and thickens. In dilated cardiomyopathy the ventricles enlarge and weaken.

People who have HCM may have a range of symptoms. People may be asymptomatic, or may have fatigue, leg swelling, and shortness of breath. It may also result in chest pain or fainting. Symptoms may be worse when the person is dehydrated. Complications may include heart failure, an irregular heartbeat, and sudden cardiac death.

HCM is most commonly inherited from a person's parents in an autosomal dominant pattern. It is often due to mutations in certain genes involved with making heart muscle proteins. Other inherited causes of left ventricular hypertrophy may include Fabry disease, Friedreich's ataxia, and certain medications such as tacrolimus. Other considerations for causes of enlarged heart are athlete's heart and hypertension (high blood pressure). Making the diagnosis of HCM often involves a family history or pedigree, an electrocardiogram, echocardiogram, and stress testing. Genetic testing may also be done. HCM can be distinguished from other inherited causes of cardiomyopathy by its autosomal dominant pattern, whereas Fabry disease is X-linked and Friedreich's Ataxia is inherited in an autosomal recessive pattern.

Treatment may depend on symptoms and other risk factors. Medications may include the use of beta blockers or disopyramide. An implantable cardiac defibrillator may be recommended in those with certain types of irregular heartbeat. Surgery, in the form of a septal myectomy or heart transplant, may be done in those who do not improve with other measures. With treatment, the risk of death from the disease is less than one percent per year.

### (ii) Dilated Cardiomyopathy (DCM)

Dilated cardiomyopathy (DCM) is a condition in which the heart becomes enlarged and cannot pump blood effectively. Symptoms vary from none to feeling tired, leg swelling, and shortness of breath. It may also result in chest pain or fainting. (DCM-2) [2] Complications can include heart failure, heart valve disease, or an irregular heartbeat.

Causes include genetics, alcohol, cocaine, certain toxins, complications of pregnancy, and certain infections. Coronary artery disease and high blood pressure may play a role, but are not the primary cause. In many cases the cause remains unclear. It is a type of cardiomyopathy, a group of diseases that primarily affects the heart muscle. The diagnosis may be supported by an electrocardiogram, chest X-ray, or echocardiogram.

In those with heart failure, treatment may include medications in the ACE inhibitor, beta blocker, and diuretic families. A low salt diet may also be helpful. In those with certain types of irregular heartbeat, blood thinners or an implantable cardioverter defibrillator may be recommended. Cardiac resynchronization therapy (CRT) may be necessary. If other measures are not effective a heart transplant may be an option in some.

(iii) Restrictive Cardiomyopathy (RCM)

Restrictive cardiomyopathy (RCM) is a form of cardiomyopathy in which the walls of the heart are rigid (but not thickened). Thus the heart is restricted from stretching and filling with blood properly. It is the least common of the three original subtypes of cardiomyopathy: hypertrophic, dilated, and restrictive. In restrictive cardiomyopathy the ventricle stiffens. It should not be confused with constrictive pericarditis, a disease which presents similarly but is very different in treatment and prognosis.

### (iv) Arrhythmogenic Right Ventricular Dysplasia ARVCM)

Arrhythmogenic cardiomyopathy (ACM), arrhythmogenic right ventricular dysplasia (ARVD), or arrhythmogenic right ventricular cardiomyopathy (ARVC), most commonly is an inherited heart disease.

ACM is caused by genetic defects of the parts of heart muscle (also called myocardium or cardiac muscle) known as desmosomes, areas on the surface of heart muscle cells which link the cells together. The desmosomes are composed of several proteins, and many of those proteins can have harmful mutations.

ARVC can also develop in intense endurance athletes in the absence of desmosomal abnormalities. Exercise-induced ARVC cause possibly is a result of excessive right ventricular wall stress during high intensity exercise.

The disease is a type of non-ischemic cardiomyopathy that primarily involves the right ventricle, though cases of exclusive left ventricular disease have been reported. It is characterized by hypokinetic areas involving the free wall of the ventricle, with fibrofatty replacement of the myocardium, with associated arrhythmias often originating in the right ventricle. The nomenclature ARVD is currently thought to be inappropriate and misleading as ACM does not involve dysplasia of the ventricular wall. Cases of ACM originating from the left ventricle led to the abandonment of the name ARVC.

ACM can be found in association with diffuse palmoplantar keratoderma, and woolly hair, in an autosomal recessive condition called Naxos disease, because this genetic abnormality can also affect the integrity of the superficial layers of the skin most exposed to pressure stress. ACM is an important cause of ventricular arrhythmias in children and young adults. It is seen predominantly in males, and 30-50% of cases have a familial distribution.

### (iv) Takotsubo Cardiomyopathy (Broken Heart Syndrome)

Takotsubo cardiomyopathy or Takotsubo syndrome (TTS), also known as stress cardiomyopathy, is a type of non-ischemic cardiomyopathy in which there is a sudden temporary weakening of the muscular portion of the heart. It usually appears after a significant stressor, either physical or emotional; when caused by the latter, the condition is sometimes called broken heart syndrome. Examples of physical stressors that can cause TTS are sepsis, shock, and pheochromocytoma, and emotional stressors include bereavement, divorce, or the loss of a job. Reviews suggest that of patients diagnosed with the condition, about 70-80% recently experienced a major stressor, including 41-50% with a physical stressor and 26-30% with an emotional stressor. TTS can also appear in patients who have not experienced major stressors.

The pathophysiology is not well understood, but a sudden massive surge of catecholamines such as adrenaline and norepinephrine from extreme stress or a tumor secreting these chemicals is thought to play a central role. Excess catecholamines, when released directly by nerves that stimulate cardiac muscle cells, have a toxic effect and can lead to decreased cardiac muscular function or “stunning”. Further, this adrenaline surge triggers the arteries to tighten, thereby raising blood pressure and placing more stress on the heart, and may lead to spasm of the coronary arteries that supply blood to the heart muscle. This impairs the arteries from delivering adequate blood flow and oxygen to the heart muscle. Together, these events can lead to congestive heart failure and decrease the heart's output of blood with each squeeze.

Takotsubo cardiomyopathy occurs worldwide. The condition is thought to be responsible for 2% of all acute coronary syndrome cases presenting to hospitals. Although TTS has generally been considered a self-limiting disease, spontaneously resolving over the course of days to weeks, contemporary observations show that “a subset of TTS patients may present with symptoms arising from its complications, e.g. heart failure, pulmonary oedema, stroke, cardiogenic shock, or cardiac arrest”. This does not imply that rates of shock/death of TTS are comparable to those of acute coronary syndrome (ACS), but that patients with acute complications may co-occur with TTS. These cases of shock and death have been associated with the occurrence of TTS secondary to an enciting physical stressor such as hemorrhage, brain injury sepsis, pulmonary embolism or severe COPD. It occurs more commonly in postmenopausal women. The name “takotsubo” comes from the Japanese word takotsubo “octopus trap”, because the left ventricle of the heart takes on a shape resembling an octopus trap when affected by this condition.

A study published in the Journal of the American Heart Association in October 2021 found a steady annual increase in takotsubo cardiomyopathy among both women and men from 2006 to 2017, with the sharpest increases among women 50 and older.

(v) Ischemic CM—Coronary infarct (ICM)

Ischemic cardiomyopathy is a type of cardiomyopathy caused by a narrowing of the coronary arteries which supply blood to the heart. Typically, patients with ischemic cardiomyopathy have a history of acute myocardial infarction, however, it may occur in patients with coronary artery disease, but without a past history of acute myocardial infarction. This cardiomyopathy is one of the leading causes of sudden cardiac death. The adjective ischemic means characteristic of, or accompanied by, ischemia-local anemia due to mechanical obstruction of the blood supply.

According to some embodiments, the TDFRPs described herein are used to treat likely or existing myocardial injury due to an unanticipated occurrence, such as a myocardial infarct, or to prevent further damage from an ongoing condition associated with myocardial injury such as certain congenital conditions or in the wake of an infection.

According to some embodiments, therapeutic uses include, but are no limited to, untreatable chronic conditions that result in cardiomyopathy. According to further embodiments, such untreatment conditions include, but are not limited to, such as autoimmune disease, pulmonary insufficiency, haemochromatosis, Anderson-Fabry disease, glycogen storage disease, medications for treating chronic conditions for which a side effect is cardiomyopathy, Gaucher's disease, Hurler's disease, Hunter's disease, diabetes mellitus, hyper and hypothyroidism, hyperparathyroidism, and pheochromocytoma.

According to another aspect, the disclosure features a method of preventing a condition that is associated with myocardial injury, the method comprising administering to a subject in need of treatment at least one tissue differentiation factor related polypeptide (TDFRP).

According to some embodiments, TDFPRs are used prophylactically, for example to prevent damage from a planned event such as the use of doxorubicin for chemotherapy or prior to a major operative procedure where either a long period under anesthesia is anticipated and/or open-heart surgery where a cardio-pulmonary pump may or will be used.

Accordingly, in some embodiments, the disclosure features a method of providing protection for an ischemic event in a subject, wherein the subject is undergoing a chemotherapy regimen, or will undergo chemotherapy regimen where a cardiotoxic agent is used, the method comprising administering to a subject in need of treatment at least one tissue differentiation factor related polypeptide (TDFRP).

Accordingly, in some embodiments, the disclosure features a method of providing protection for an ischemic event in a subject, wherein the subject will undergo open-heart surgery, the method comprising administering to a subject in need of treatment at least one tissue differentiation factor related polypeptide (TDFRP).

### B. Source of Cardiomyocytes

Isolated cardiomyocyte cultures or cardiomyocytes present within cardiac tissue or the heart organ may be treated with the TDFRPs described herein. In addition to human, a cardiomyocyte according to the methods of the present disclosure may be derived from any mammal, including for example, a pig, mouse, or non-human primate monkey. Cardiomyocytes amenable to treatment may be of any maturity state, and thus include neonatal cardiomyocytes, stem cells, cells committed to differentiate to cardiomyocytes, a myocyte derived from a non-heart muscle, a myoblast, or adult cardiomyocytes.

Mammals having a cardiac disorder may be directly administered a TDFRP. Alternatively, cardiomyocytes, or cardiac tissue may be isolated from a mammal, treated with a TDFRP ex vivo and transplanted back into the patient. Such cardiomyocytes, cardiac tissue, or heart may also derive from a donor mammal for transplantation into a recipient mammal. The donor and recipient may or may not be from the same species. For example, a pig heart treated according to the methods of this disclosure may be transplanted into a human recipient.

### C. Biological Effect

In various embodiments, suitable in vitro or in vivo assays are performed to determine the effect of a specific TDFRP-based therapeutic.

In various specific embodiments, in vitro assays can be performed with representative cells of the type(s) involved in the patient's disorder, to determine if a given TDFRP-based therapeutic exerts the desired effect upon the cell type(s). Compounds for use in therapy can be tested in suitable animal model systems including, but not limited to rats, mice, chicken, cows, monkeys, rabbits, and the like, prior to testing in human subjects. Similarly, for in vivo testing, any of the animal model system known in the art can be used prior to administration to human subjects.

In some embodiments, a more complex in vitro system that better mimics the in vivo setting may be used for testing the efficacy of a compound or analog, e.g., TDFRP compound

### (i) In Vitro Apoptosis Assays

A number of assays may be used to measure cardiomyocyte apoptosis in vitro, including for example DNA laddering, cell death ELISA, flow cytometry for DNA content, TUNEL assay, the measurement of caspase activity, or the detection of surrogate markers of apoptosis by Western or Northern analysis, or alternatively by RT-PCR.

For determination of DNA laddering, cellular DNA is extracted with phenol: chloroform, treated with RNase, 32P-labeled, and then visualized by electrophoresis in 1.8% agarose gel.

In the TUNEL assay, terminal deoxynucleotidyl transferase is used to incorporate digoxigenin-labeled dUTP into 3′-OH DNA ends generated by DNA fragmentation and detected by counterstaining with peroxidase labeled anti-digoxigenin mAb (ApoTag, Intergen).

Flow cytometry may also be used to measure DNA content in cardiomyocytes. Cells are fixed with 80% ethanol and stained with propidium iodide after RNase treatment. Apoptotic cells register as containing less than the diploid DNA quantity (2N).

Histone-associated DNA fragments are quantified by cell death ELISA as described in the manufacturer's protocol. DNA fragmentation data are corrected for background and normalized to the result with normoxic cardiomyocytes.

Optionally, apoptosis may also be measured by the protein or gene expression, or alternatively, by the activity of surrogate markers of apoptosis including, for example, caspase activity. Caspase-3, -8, and -9 activity may be examined by using the caspase colorimetric assay kit from R&D Systems according to the manufacturer's protocol. Briefly, cells are scraped, collected, washed with cold PBS, and lysed in cold lysis buffer. Lysates are incubated on ice for 10 min and centrifuged (10,000×g, 1 min). The supernatants are removed and assayed for caspase activity. The specific peptide substrates used for each individual caspase are DEAD-pNA, LEHD-pNA, and IETD-pNA for caspase-3, -9, and -8, respectively. Release of the pNA cleavage product is quantitated in a microplate reader (Bio-Rad) at a wavelength of 405 nm.

Optionally, several overlapping assays may be performed to ensure that cell death is apoptotic (nuclear morphology and DNA laddering), that dying cells are in fact CM (TUNEL/double staining/confocal), and that quantitative comparisons of different populations can be made (ELISA for histone-associated DNA fragments/FACS for DNA content). General assays of cell viability (e.g., trypan blue exclusion, MTT, etc.) can also be used to assess overall cytoprotection (i.e., not just protection from apoptosis).

### (ii) Determination of Apoptosis In Vivo

To determine apoptosis in vivo, TUNEL staining can be performed using Apoptag (Intergen) according to the manufacturer's instructions, with Hoechst 33258 (Sigma) nuclear counter-staining. Alternatively, DNA laddering can also be used to detect apoptosis of cardiomyocytes. Fresh tissues (without TTC staining) are microdissected under UV light into ischemic and non-ischemic regions and processed simultaneously. Tissues from each region are lysed and DNA is prepared, labeled with [α-32P] dCTP, and subjected to electrophoresis and autoradiography as described by Vazquez-Jimenez et al (J. Am. Coll. Cardiol., 2001). Optionally, apoptosis of cardiomyocytes may further be determined by the detection of surrogate markers of apoptosis, such caspase activation, for example.

(iii) Determination of Inflammation

Cardiac inflammation may be determined by the detection of pro-inflammatory markers, the release of pro-inflammatory molecules (MCP-1), or by the activation of pro-inflammatory signaling in cardiomyocytes. NF-κB activation correlates with cardiac inflammation and may be determined both in vitro (by Western or Northern analysis for example) or in vivo (as measured by immunohistochemical methods). NF-κB activation is typically demonstrated by phosphorylation and degradation of IκB, nuclear translocation of the p65 NF-κB subunit, or increased mRNA for the NF-κB-dependent transcripts, VCAM-1 and ICAM-1.

Alternatively, overall morphology of cardiac tissue and the detection of infiltration of inflammatory cells, such as leukocytes (by immunohistochemical methods) also demonstrate the presence of inflammation in cardiac tissue.

### IV. PHARMACEUTICAL COMPOSITIONS

The pharmaceutical compositions of the disclosure typically contain a therapeutically effective amount of a compound described herein. Those skilled in the art will recognize, however, that a pharmaceutical composition may contain more than a therapeutically effective amount, such as in bulk compositions, or less than a therapeutically effective amount, that is, individual unit doses designed for multiple administration to achieve a therapeutically effective amount. Typically, the composition will contain from about 0.01-95 wt % of active agent, including, from about 0.01-30 wt %, such as from about 0.01-10 wt %, with the actual amount depending upon the formulation itself, the route of administration, the frequency of dosing, and so forth. According to one embodiment, a composition suitable for an oral dosage form, for example, may contain about 5-70 wt %, or from about 10-60 wt % of active agent.

TDFRPs may be administered to patients or experimental animals with a pharmaceutically-acceptable diluent, carrier, or excipient, in unit dosage form. Conventional pharmaceutical practice may be employed to provide suitable formulations or compositions to administer such compositions to patients or experimental animals. Although intravenous administration is preferred, any appropriate route of administration may be employed, for example, parenteral, subcutaneous, intramuscular, intracranial, intraorbital, ophthalmic, intraventricular, intracapsular, intraspinal, intracisternal, intraperitoneal, intranasal, aerosol, oral, or topical (e.g., by applying an adhesive patch carrying a formulation capable of crossing the dermis and entering the bloodstream) administration. Therapeutic formulations may be in the form of liquid solutions or suspensions; for oral administration, formulations may be in the form of tablets or capsules; and for intranasal formulations, in the form of powders, nasal drops, or aerosols. Any of the above formulations may be a sustained-release formulation.

Methods well known in the art for making formulations are found in, for example, “Remington's Pharmaceutical Sciences.” Formulations for parenteral administration may, for example, contain excipients, sterile water, or saline, polyalkylene glycols such as polyethylene glycol, oils of vegetable origin, or hydrogenated napthalenes. Sustained-release, biocompatible, biodegradable lactide polymer, lactide/glycolide copolymer, or polyoxyethylene-polyoxypropylene copolymers may be used to control the release of the compounds. Other potentially useful parenteral delivery systems for administering molecules of the disclosure include ethylene-vinyl acetate copolymer particles, osmotic pumps, implantable infusion systems, and liposomes. Formulations for inhalation may contain excipients, for example, lactose, or may be aqueous solutions containing, for example, polyoxyethylene-9-lauryl ether, glycocholate and deoxycholate, or may be oily solutions for administration in the form of nasal drops, or as a gel.

### A. Unit Dosage

According to some embodiments, it is advantageous to formulate oral or parenteral compositions in dosage unit form for ease of administration and uniformity of dosage. Dosage unit form as used herein refers to physically discrete units suited as unitary dosages for the subject to be treated; each unit containing a predetermined quantity of active compound calculated to produce the desired therapeutic effect in association with the required pharmaceutical carrier. The specification for the dosage unit forms of the disclosure are dictated by and directly dependent on the unique characteristics of the active compound and the particular therapeutic effect to be achieved, and the limitations inherent in the art of compounding such an active compound for the treatment of individuals.

### B. Gene Therapy

In yet another embodiment of the disclosure, the polypeptide may be administered by administering an expression vector encoding the polypeptide to the mammal.

TDFRP polypeptides described herein may also be administered by somatic gene therapy. Expression vectors for gene therapy (e.g., plasmids, artificial chromosomes, or viral vectors, such as those derived from adenovirus, retrovirus, poxvirus, or herpesvirus) carry a TDFRP-encoding DNA under the transcriptional regulation of an appropriate promoter. The promoter may be any non-tissue-specific promoter known in the art (for example, an SV-40 or cytomegalovirus promoter). Alternatively, the promoter may be a tissue-specific promoter, such as a striated muscle-specific, an atrial or ventricular cardiomyocyte-specific (e.g., as described in Franz et al., Cardiovasc. Res. 35:560-566, 1997), or an endothelial cell-specific promoter. The promoter may be an inducible promoter, such as the ischemia-inducible promoter described in Prentice et al. (Cardiovasc. Res. 35:567-574, 1997).

The expression vector may be administered as naked DNA mixed with or conjugated to an agent to enhance the entry of the DNA into cells, e.g., a cationic lipid such as LIPOFECTIN, LIPOFECTAMINE (Gibco/BRL, Bethesda, Md.), DOTAP™ (Boeringer-Mannheim, Indianapolis, Ind.) or analogous compounds, liposomes, or an antibody that targets the DNA to a particular type of cell, e.g., a cardiomyocyte or an endothelial cell. The method of administration may be any of those described herein. In particular, DNA for somatic gene therapy has been successfully delivered to the heart by intravenous injection, cardiac perfusion, and direct injection into the myocardium (e.g., see Losordo et al., Circulation 98:2800-2804, 1998; Lin et al., Hypertension 33:219-224, 1999; Labhasetwar et al., J. Pharm. Sci. 87:1347-1350, 1998; Yayama et al., Hypertension 31:1104-1110, 1998).

All publications and patent applications cited in this specification are herein incorporated by reference in their entirety for all purposes as if each individual publication or patent application were specifically and individually indicated to be incorporated by reference for all purposes. The publications discussed herein are provided solely for their disclosure prior to the filing date of the present application. Nothing herein is to be construed as an admission that the inventors described herein are not entitled to antedate such disclosure by virtue of prior disclosure or for any other reason.

### EXAMPLES

The following examples are intended to be non-limiting illustrations of certain embodiments of the present disclosure. All references cited are hereby incorporated herein by reference in their entireties.

### Example 1. Cell Starvation Via Serum Withdrawal LED to Ischemia and the Reduction of Phosphorylated AKT

The examples described herein valuated the effect of TDFRP's on the physiology of cardiomyocytes in vitro. Phosphorylation of cellular AKT at Ser473 is known to promote cardiomyocyte survival, function, and contractility. Cellular enzyme Caspase-3 is responsible for apoptosis via both apoptosis pathways. Chemotherapeutic agent doxorubicin blocks the phosphorylation of AKT used to treat a diverse number of cancers, but one significant side effect is decreased heart function. In cardiomyocytes, doxorubicin hinders the phosphorylation of AKT and stimulates Caspase-3. Lipopolysaccharides (LPS) are endotoxins that promote inflammation and apoptosis in cardiomyocytes.

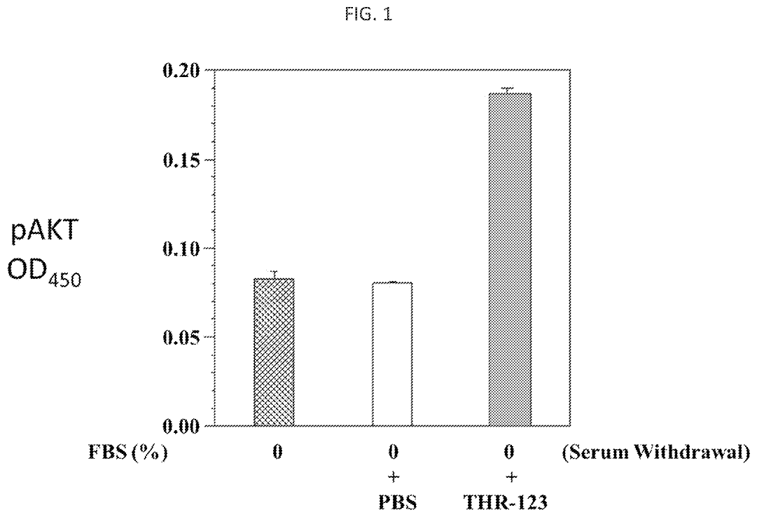

Primary neonatal rat cardiomyocytes (Cell Applications) were cultured in medium with 10% growth supplement (Cell Applications) for 24 hours at 37 °C, 5% CO2. Cells were then starved of growth supplement for 12 hours to create ischemic injury. Controls received medium alone. Cells were incubated with culture medium alone, BMP-7 (143 nM) or SEQ ID No. 1 (100 μM) for 60 h. In FIG. 1, Akt phosphorylation at Serine 473 (Cell Signaling Technology) is depressed due to media starvation and was not affected by the addition of PBS. The addition of THR-123 (SEQ ID NO: 1) significantly increased Akt Ser473 phosphorylation (p<0.01).

### Example 2. The Effect of TDFRPs on the Depression of pAKT Due to Doxorubicin

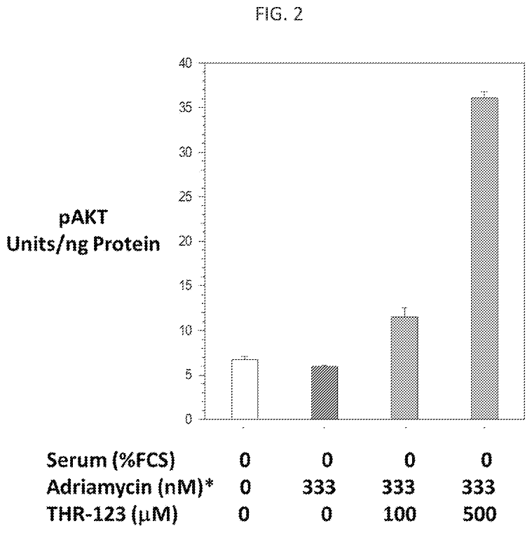

Neonatal rat cardiomyocytes (CM) were pre-treated with 0.33 μM doxorubicin for 24 h, followed by treatment with BMP-7 (143 nM) or THR-123 (SEQ ID NO: 1) at 100 and 500 μM for 60 h. The TDFRP peptide significantly increased cellular Akt phosphorylation (p<0.01) compared to doxorubicin alone (FIG. 2), and inhibited doxorubicin induced Caspase-3 activity in a dose dependent manner (p<0.05) (FIG. 2).

### Example 3. The Chemotherapeutic Agent Doxorubicin Stimulated Caspase-3. The Effect of BMP-7 and TDFRPs on the Level of Caspase-3

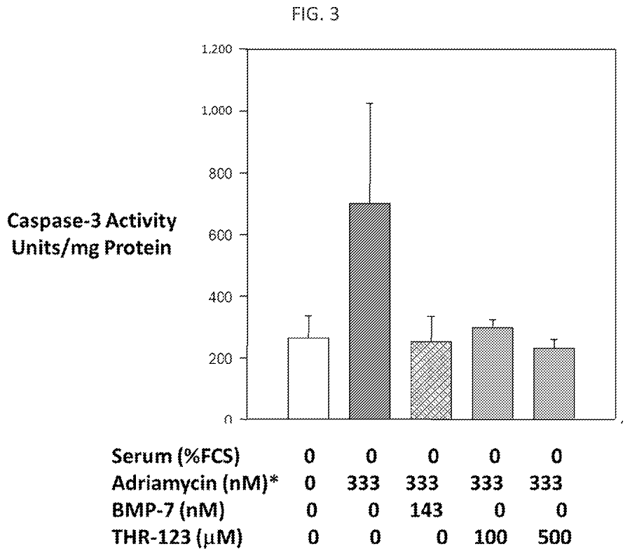

Neonatal rat cardiomyocytes (CM) were pre-treated with 0.33 μM doxorubicin for 24 h, followed by treatment with BMP-7 (143 nM) or SEQ ID No. 1 at 100 and 500 μM for 60 h. Both BMP-7 and THR-123 (SEQ ID NO: 1) significantly inhibited doxorubicin induced Caspase-3 activity (p<0.05) (FIG. 3). Caspase-3 activation is a step common to both apoptotic pathways and leads to apoptosis.

### Example 4. The Effect of TDFRP Peptide SEQ ID NO: 1 on LPS Induced Caspase-3

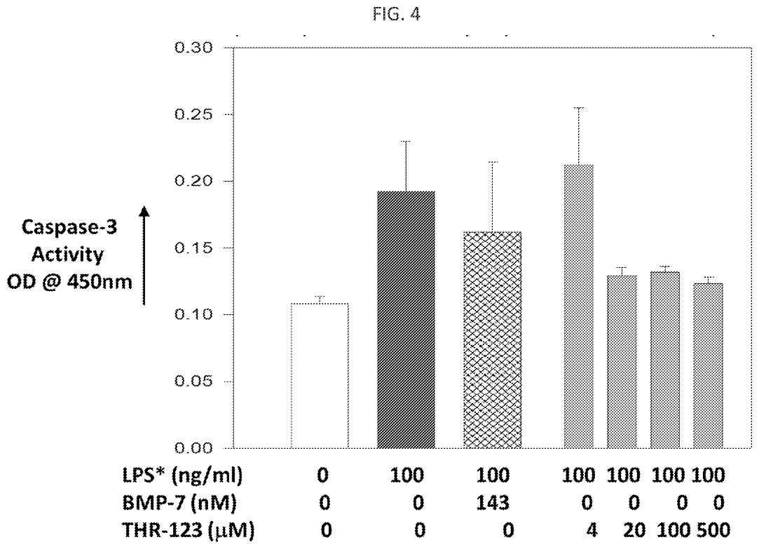

Lipopolysaccharide (LPS) induced Caspase-3 in cardiomyocytes. As shown in FIG. 4, both BMP-7 (143 nM) and THR-123 (SEQ ID NO: 1) at 4, 20, 100, and 500 μM reduced the level of LPS induced Caspase-3 in a concentration dependent manner.

### Example 5. TDFRP Peptide SEQ ID NO: 1 Reduces Inflammation in Cardiomyocytes

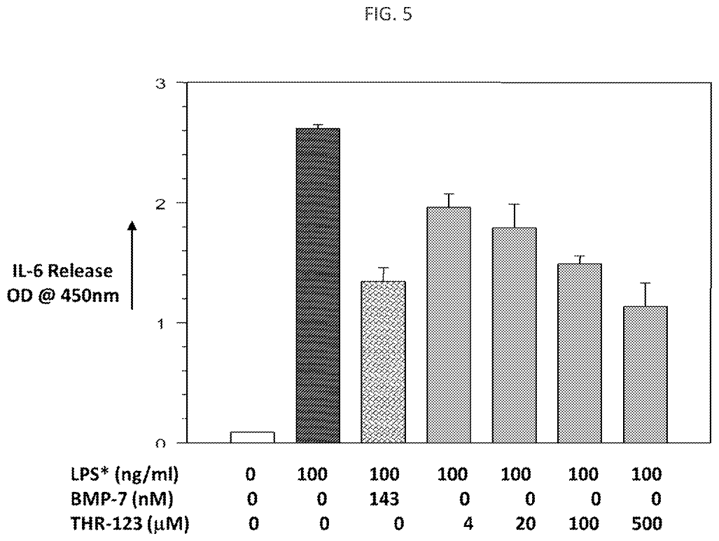

LPS induces the production of cytokine IL-6, a marker of inflammation. As shown in FIG. 5, Both BMP-7 (143 nM) and THR-123 (SEQ ID NO: 1) at 4, 20, 100, and 500 μM reduces the level of LPS induced IL-6 in a concentration dependent manner.

### Example 6. An In Vivo Study of the Effect of TDFRP Peptide THR-123 (SEQ ID NO: 1) in Ameliorating the Effects of Myocardial Ischemia

The efficacy of BMP mimetic THR-123 in ameliorating the effects of myocardial ischemia was investigated in the rat after ligation of the coronary artery. The rat LAD model is used for studying the effect of treatments for ameliorating the effect of a heart attack (coronary infarct). The Left Anterior Descending artery (LAD) supplies blood to the left ventricle. Blocking blood flow through the LAD creates ischemia in a region of the left ventricle muscle (region at risk). Releasing the blockage leads to reperfusion injury when immune cells in the recirculating blood attack injured cardiomyocytes.

Myocardial ischemia was created by ligating the left anterior descending artery (LAD), which supplies blood to the left ventricle of the heart. In the LAD infarct model the chest and the pericardium are opened to expose the LAD. A suture is placed around the LAD and loops are added to the two ends, then the suture is tightened with an over hand knot (ligation). The ligation blockage to the LAD was maintained for 20 min to simulate a coronary infarct, then released using the two loops.

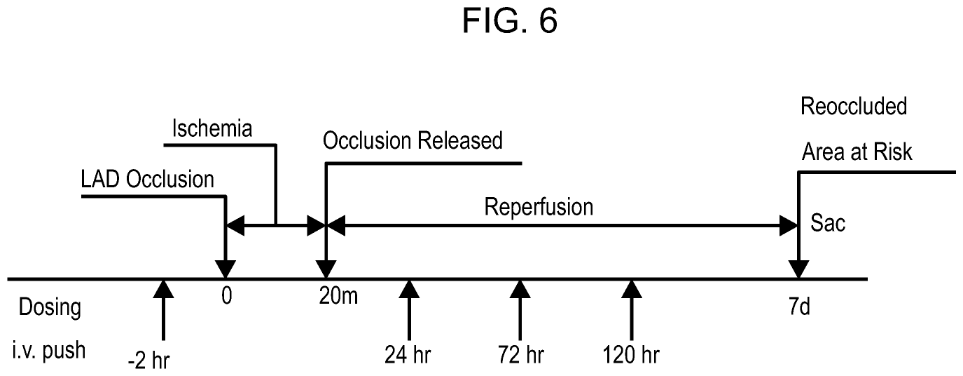

A schematic of the study is shown in FIG. 6

The animals were divided into three groups that were dosed with PBS, BMP-7, or THR-123 (SEQ ID NO: 1). Administrations were at 2 hr prior to ligation and at 24, 72 and 120 hr post ligation. On the seventh day post ligation the suture was tightened again and Methyl Blue was injected into the blood stream to stain region of the heart that the ligation did not affect (the unaffected region).

Twenty-four unmanipulated rats, sorted into three large groups by initial body weight (>300 g pre-surgery) were evaluated. Animals underwent surgery and were sorted into 3 treatment groups: Group 1: Vehicle (neg. control), PBS, equal volume i.v; Group 2: BMP-7, 160 μg/kg i.v.<5 ml/kg; Group 3: THR-123 (SEQ ID NO: 1), 10 mg/kg i.v.<5 ml/kg. The LAD was ligated to create a 20 min period of ischemia, then released for seven days during which reperfusion injury occurs (IR injury).

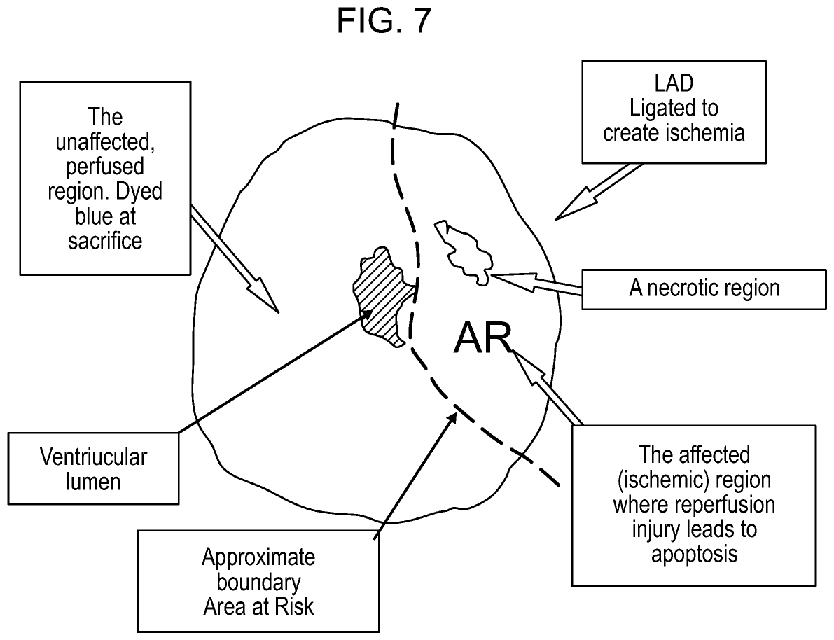

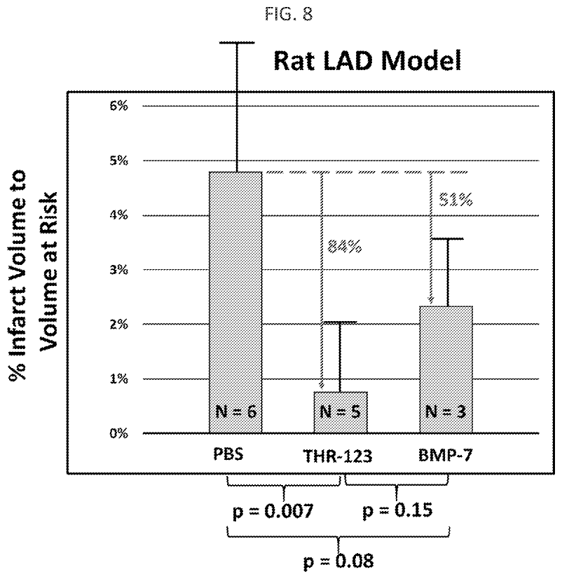

The effect of the LAD ligation was measured via histomorphometric analysis of heart slices where the necrotic areas (yellow) and the areas impacted by the LAD ligation (area at risk, i.e., not stained blue) were measured for each slice and summed over all slices to obtain a measure of the infarct volume and the volume at risk (FIG. 7). The ratio of necrotic volume to volume at risk is a measure of myocardial damage. FIG. 8 shows a plot of the myocardial damage as a function of treatment regimen: PBS alone, BMP-7 and THR-123 (SEQ ID NO: 1). As shown in FIG. 8, relative to PBS, the presence of BMP-7 reduces IR damage by 51% (p=0.08), and THR-123 (SEQ ID NO: 1) reduces IR damage by 85% (p=0.007).

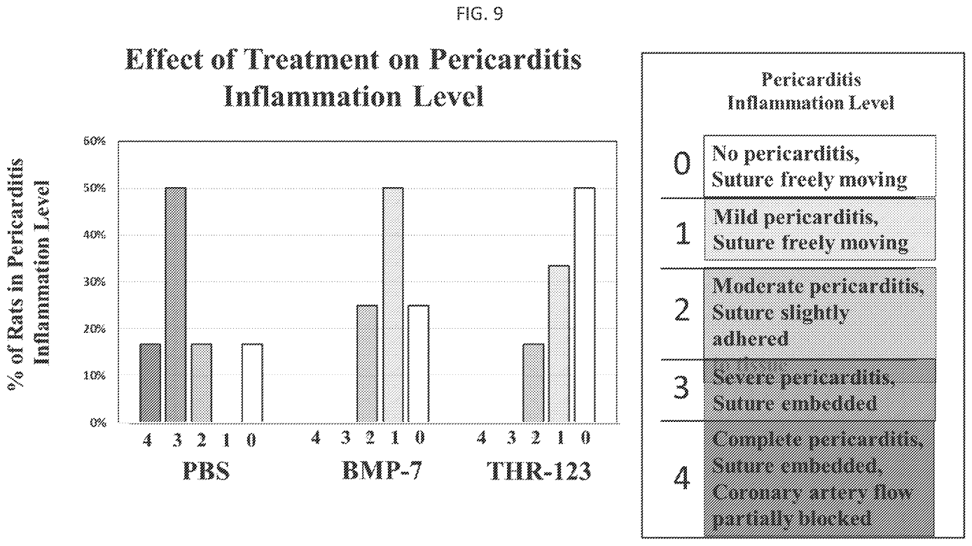

Treatment with BMP-7 and THR-123 (SEQ ID NO: 1) also reduced paracardial inflammation relative to PBS. The predominance of the hearts from the PBS treated group had more severe inflammation than hearts from the BMP-7 treated and the THR-123 (SEQ ID NO: 1) treated groups (FIG. 9).

We claim:

1. A method for treating or preventing myocardial injury, or a condition associated with myocardial injury, in a mammal, wherein the method comprises administering at least one tissue differentiation factor related polypeptide (TDFRP) to the mammal, and wherein the administering is in an amount effective to treat or prevent myocardial injury in said mammal.

2. The method of claim 1, wherein the TDFRP is selected from an amino acid sequence at least 90% identical to an amino acid sequence selected from the group consisting of: SEQ ID NO: 1, SEQ ID NO: 2, SEQ ID NO: 3, SEQ ID NO: 4, SEQ ID NO: 5, SEQ ID NO: 6, SEQ ID NO: 7, SEQ ID NO: 8, and SEQ ID NO: 9.

3. The method of claim 1, wherein the TDFRP consists of an amino acid sequence selected from the group consisting of: SEQ ID NO: 1, SEQ ID NO: 2, SEQ ID NO: 3, SEQ ID NO: 4, SEQ ID NO: 5, SEQ ID NO: 6, SEQ ID NO: 7, SEQ ID NO: 8 and SEQ ID NO: 9.

4. The method of any one of claims 1-3, wherein the mammal is a human.

5. The method of any one of claims 1-4, wherein the myocardial injury results from or is associated with an ischemia/reperfusion injury.

6. The method of claim 5, wherein the ischemia/reperfusion injury results from myocardial infarction, severe trauma, renal insufficiency, pulmonary insufficiency, arterial stenosis, open heart surgery, or doxorubicin chemotherapy.

7. The method of any one of claims 1-4, wherein the myocardial injury results from or is associated with a surgical procedure; exposure to a cardiotoxic compound; hypertension; ischemic heart disease; dilated cardiac injury; myocarditis; thyroid disease; viral infection; gingivitis; drug abuse; alcohol abuse; periocarditis; atherosclerosis; vascular disease; hypertrophic cardiomyopathy; acute myocardial infarction; left ventricular systolic dysfunction; coronary bypass surgery; starvation; an eating disorder; or a genetic defect.

8. The method of claim 7, wherein the cardiotoxic compound is an anthracycline, alcohol, or cocaine.

9. The method of claim 8, wherein the anthracyline is doxorubicin, or daunomycin.

10. The method of any one of claims 1-9, wherein the TDFRP is administered prior to the diagnosis of the myocardial injury, or the condition associated with myocardial injury in the mammal.

11. The method of any one of claims 1-9, wherein the TDFRP is administered after the diagnosis of the myocardial injury, or the condition associated with myocardial injury in the mammal.

12. The method of any one of claims 1-11, wherein the mammal is at risk for myocardial injury.

13. The method of claim 12, wherein the mammal is an individual that smokes, is obese, has been or will be exposed to a cardiotoxic compound, has or had high blood pressure, has or had ischemic heart disease, has or had a myocardial infarct, has a genetic defect that increases the risk of heart failure, has a family history of heart failure, has or had myocardial hypertrophy, has or had hypertrophic cardiomyopathy, has or had left ventricular systolic dysfunction, had coronary bypass surgery, has or had vascular disease, has or had atherosclerosis, has or had alcoholism, has or had pericarditis, has or had a viral infection, has or had gingivitis, has or had an eating disorder, has or had myocarditis, has or had a thyroid disease, or is a cocaine addict.

14. The method of any one of claims 1-13, wherein the mammal has or had a cancer.

15. The method of any one of claims 1-14, wherein administration of the TDFRP inhibits cardiomyocyte apoptosis.

16. The method of any one of claims 1-14, wherein administration of the TDFRP inhibits pericarditis inflammation levels.

17. The method of any one of claims 1-16, wherein the TDFRP is administered by a parenteral, intravenous, subcutaneous, intramuscular, intracranial, intraorbital, ophthalmic, intraventricular, intracapsular, intraspinal, intracisternal, intraperitoneal, intranasal, aerosol, oral, or topical route.

18. The method of any one of claims 1-17, further comprising the administration of an additional agent selected from the group consisting of: anti-neoplastic agents, antibiotics, vaccines, immunosuppressive agents, anti-hypertensive agents and mediators of the hedgehog signaling pathway.


SEQUENCE LISTING (18 sequences)

SEQ ID NO: 1 - protein, 16 aa, synthetic construct
  REGION 1..16: note = Synthetic
  SITE 1..16: note = N-terminal (H) and a C-terminal (OH)
CYFDDSSNVLCKKYRS

SEQ ID NO: 2 - protein, 16 aa, synthetic construct
  REGION 1..16: note = Synthetic
  SITE 1..16: note = N-terminal (H) and a C-terminal (OH)
CYYDNSSSVLCKRYRS

SEQ ID NO: 3 - protein, 16 aa, synthetic construct
  REGION 1..16: note = Synthetic
  SITE 1..16: note = N-terminal (H) and a C-terminal (OH)
CYYDNSSSVLCKKYRS

SEQ ID NO: 4 - protein, 16 aa, synthetic construct
  REGION 1..16: note = Synthetic
  SITE 1..16: note = N-terminal (H) and a C-terminal (OH)
CYFDDSSQVICKKYRS

SEQ ID NO: 5 - protein, 16 aa, synthetic construct
  REGION 1..16: note = Synthetic
  SITE 1..16: note = N-terminal (H) and a C-terminal (OH)
CYFDDSSSVLCKKYRS

SEQ ID NO: 6 - protein, 16 aa, synthetic construct
  REGION 1..16: note = Synthetic
  SITE 1..16: note = N-terminal (H) and a C-terminal (OH)
CYFDDSSNVLCKKYRN

SEQ ID NO: 7 - protein, 16 aa, synthetic construct
  REGION 1..16: note = Synthetic
  SITE 1..16: note = N-terminal (H) and a C-terminal (NH2)
CYFDDSSNVLCKKYRS

SEQ ID NO: 8 - protein, 16 aa, synthetic construct
  REGION 1..16: note = Synthetic
  SITE 1..16: note = N-terminal (H) and a C-terminal (OH)
CYYDDSSNVLCKKYRS

SEQ ID NO: 9 - protein, 16 aa, synthetic construct
  REGION 1..16: note = Synthetic
  SITE 1..16: note = N-terminal (H) and a C-terminal (OH)
CYYDDSSSVLCKKYRS

SEQ ID NO: 10 - protein, 16 aa, synthetic construct
  REGION 1..16: note = Synthetic
CYFDDSSNVLCKKYRS

SEQ ID NO: 11 - protein, 16 aa, synthetic construct
  REGION 1..16: note = Synthetic
CYYDNSSSVLCKRYRS

SEQ ID NO: 12 - protein, 16 aa, synthetic construct
  REGION 1..16: note = Synthetic
CYYDNSSSVLCKKYRS

SEQ ID NO: 13 - protein, 16 aa, synthetic construct
  REGION 1..16: note = Synthetic
CYFDDSSQVICKKYRS

SEQ ID NO: 14 - protein, 16 aa, synthetic construct
  REGION 1..16: note = Synthetic
CYFDDSSSVLCKKYRS

SEQ ID NO: 15 - protein, 16 aa, synthetic construct
  REGION 1..16: note = Synthetic
CYFDDSSNVLCKKYRN

SEQ ID NO: 16 - protein, 16 aa, synthetic construct
  REGION 1..16: note = Synthetic
CYFDDSSNVLCKKYRS

SEQ ID NO: 17 - protein, 16 aa, synthetic construct
  REGION 1..16: note = Synthetic
CYYDDSSNVLCKKYRS

SEQ ID NO: 18 - protein, 16 aa, synthetic construct
  REGION 1..16: note = Synthetic
CYYDDSSSVLCKKYRS